(19) United States
(12) Patent Application Publication
(10) Pub. No.: US 2008/0102114 A1
(43) Pub. Date: May 1, 2008

(54) Microparticles and Nanoparticles for the Transmucosal Delivery of Therapeutic and Diagnostic Agents

(76) Inventors: Panduranga Rao Koritala, Naperville, IL (US); Srirajasekhar Koritala, Naperville, IL (US)

Correspondence Address: ROPES & GRAY LLP, PATENT DOCKETING 39/41, ONE INTERNATIONAL PLACE, BOSTON, MA, 02110-2624, US

(21) Appl. No.: 11/578,885

(22) PCT Filed: Apr. 25, 2005
(86) PCT No.: PCT/US05/14268

§ 371 Date: Sep. 18, 2007

Related U.S. Application Data
(60) Provisional application No. 60/651,486, filed on Apr. 23, 2004.

(51) Int. Cl. A61K 9/51; A61K 38/28; A61K 9/48

(52) U.S. Cl. 424/456; 424/490; 424/499; 514/3

(57) ABSTRACT

The invention relates to compositions and methods for the administration of therapeutic and/or diagnostic agents such as polypeptides to a mammal, and in particular, compositions suitable for oral administration. The invention provides polymeric particles, and in particular, nano/microparticles such as, but not limited to, microspheres and nanospheres, as well as methods of synthesizing them. The invention also provides methods of increasing the serum concentration of a therapeutic agent such as a polypeptide by orally administering polymeric particles comprising the therapeutic agent. The compositions of the invention allow the absorption of polypeptides through intestinal mucosa and intestinal cells and into the bloodstream of a mammal. The invention further provides a method of treating type II diabetes through the oral administration of compositions comprising insulin and also provides a related glucose-responsive insulin delivery system.

### CROSS-REFERENCE TO RELATED APPLICATIONS

This application claims the benefit of the filing date of U.S. application Ser. No. 10/831,465, filed Apr. 23, 2004, entitled “NANO AND MICROSPHERES FOR ORAL DELIVERY OF THERAPEUTIC AGENTS.” The entire teachings of the referenced application are incorporated by reference herein.

### BACKGROUND OF THE INVENTION

Significant advances in biotechnology have resulted in the discovery of large number of therapeutic compounds including peptides and proteins. However, the primary mode of administration of peptides or polypeptides to individuals is largely confined to injections. Injections can be painful, may result in inflammatory responses and scarring at the site of injection, and commonly result in noncompliance especially among younger patients.

Although non-invasive delivery systems are desirable, general problems exist in developing non-invasive delivery systems for high molecular weight compounds such as proteins. Normally such molecules are not taken up by the body if administered to the gastrointestinal tract, the buccal mucosa, the rectal mucosa or the vaginal mucosa, or if given as an intranasal system, and may be degraded by proteases such as those of the digestive system. Absorption enhancers, which aid in the transport of macromolecules across the mucosal surfaces, such as the intestinal mucosal surfaces, are sometimes coadministered with the macromolecules. Furthermore, enzyme inhibitors have been used to facilitate transport of therapeutic molecules and prevent degradation by proteases of the digestive system. However, penetration enhancers and enzyme inhibitors can have adverse effects on the intestinal membranes or digestive process.

Accordingly, there is a need to provide effective methods and compositions for the absorption of high molecular weight material such as proteins and peptides across biological membranes. The invention provides polymeric particles and delivery vehicles, and methods of making and using them, capable of delivering therapeutic and diagnostic agents, including proteins and peptides, across mucosal surfaces and into the bloodstream of mammal.

### SUMMARY OF THE INVENTION

The invention generally relates to polymeric particles comprising therapeutic or diagnostic agents, and in particular to nano/microparticles. The polymeric particles of the invention are preferably in the form of nano/microparticles. The terms nano/microparticles include nano/microspheres and nano/microcapsules. The compositions according to the present disclosure, preferably in the form of nano/microspheres, provide improved transport of agents including peptides and proteins, and improved presentation of therapeutic agents such as vaccines to mucosal surfaces.

One aspect of the invention relates to compositions comprising chitosan nanospheres and microspheres (“SparkCORAL™ nano/microspheres”). Another aspect relates to methods of making the compositions and methods of using them in therapeutic and diagnostic applications. In some embodiments, the microparticles and nanoparticles are packaged in a stimuli-responsive gelatin capsule for regulated delivery to a mammal.

The invention further provides synthetic and natural polymeric, cationic nano/microspheres for used as protein-delivery vehicles. The compositions are suitable for delivering agents such as polypeptides to mucosal membranes including those of the intestine or lungs. Natural polymers that may be used in the invention include chitosan or modified chitosan, preferably in the form of cross-linked nano/microparticles, such as spheres, discs and tubes.

In some embodiments, the nano/microparticles of the invention exhibit pH dependent swelling. This may be achieved by the introduction or presence of basic or acidic pendent groups in the polymer.

Some aspects of the invention provide polymeric particles that allow a pH-dependent release of therapeutic and diagnostic agents. In one embodiment, the pH-dependent release of the agent is coupled to one or more enzymes which alter the pH of the particle in the presence of a substrate. In one embodiment, glucose oxidase, catalase and insulin are entrapped in, dispersed within, covalently bonded to, or embedded within the nano/microspheres to generate a glucose-responsive insulin delivery vehicle. When exposed to physiological fluids, glucose diffuses into the hydrogel, glucose oxidase catalyzes the conversion of glucose to gluconic acid, causing swelling of the pH sensitive spheres and subsequent insulin release.

Another aspect of the invention provides a delivery device for targeted delivery of the polymeric particles to a mucosal membrane. The invention provides a multiple unit carrier system for the specific delivery of SparkCORAL™ nano/microspheres, or other polymeric particles, to intestinal membranes. In one embodiment, the multiple-unit carrier system comprises alginate-coated gelatin capsules as safe candidates for the oral delivery of therapeutic/diagnostic agents to mammals including humans, to carry SparkCORAL™ nano/microspheres containing bioactive agents such as peptides and proteins and deposit them selectively in the intestine where the therapeutic action or drug absorption is desired. The multiple unit carrier system comprising polymer-coated gelatin capsules is useful as it provides routine use of the oral route of drug delivery for protein and peptide drugs.

The nano/microparticles of the invention typically comprise a therapeutic or diagnostic agent dispersed throughout. In some embodiments, the microspheres or nanospheres of the invention include at least one therapeutic agent, such as protein, antigens, antibodies, antibody fragments, enzymes, vaccines, enzyme inhibitors, DNA, RNA, antisense oligonucleotides and other regulatory nucleic acids such as siRNAs and shRNAs, genes, gene constructs, or combinations thereof. Such agents include a vaccine immobilized or entrapped within microspheres or nanospheres. Certain preferred microspheres or nanospheres of the present invention may comprise a hormone such as insulin.

One aspect of the invention also provides a method of improving the hydrophobic/hydrophilic balance and buffering effect for protection of polymeric particles from lysosomal proteolysis. In one embodiment, chitosan nano/microspheres are coated with a hydrophobic agent, such as a polycaprolactone, and subsequently with a hydrophilic agent, such as chitosan. In preferred embodiments, the hydrophobic and hydrophilic agents are polymers, and in particular, biodegradable polymers.

The invention further relates to improved methods of preparing polymeric particles, and in particular micro- and nanoparticles such as microspheres and nanospheres. One aspect provides a method of preparing a plurality of nanospheres or microspheres, comprising the steps of: cross-linking a polymer, such as chitosan, using a dialdehyde, such as organic phase glutaraldehyde under conditions suitable for formation of nanospheres or microspheres. Some embodiments further comprise blocking residual or free aldehyde groups in polymer chitosan and/or further crosslinking the crosslinked chitosan with an agent that does not crosslink amino groups, such as epichlorohydrin, diisocyanate, or blocked diisocyanate.

Other methods provided by the invention for the synthesis of polymeric microparticles and nanoparticles comprise aminating the microparticles and nanoparticles with a suitable aminating agent. Exemplary aminating agents include ethylenediamine, spennine, 1,12-diaminododecane, 4,4″-diaminodicyclohexyl methane, 4,4″-diaminobibenzyl, jeffamine, cholaminochloride hydrochloride, and cholestryamine.

Other methods provided by the invention for the synthesis of polymeric microparticles and nanoparticles comprise coating the microparticles and nanoparticles with a hydrophobic polymer, such as a polycaprolactone. The nanospheres or microspheres may further be coated with a hydrophilic polymer, such as with a chitosan or a modified chitosan. A related aspect of the invention provides polymeric particles, including nanospheres and microspheres, prepared according to any of the methods described herein.

Some aspects of the invention provide a drug delivery system comprising a plurality of microspheres and/or nanospheres along with a suitable pharmaceutical carrier. In some preferred embodiments, the drug delivery system further includes a glucose responsive system comprising immobilized glucose oxidase, catalase and insulin.

The invention further provides nano/microparticles for the manufacture of medicaments to treat disorders. Any methods disclosed herein for treating, preventing or aiding in the prevention of a disorder, such as type II diabetes, by administering nano/microparticles to an individual may be applied to the use of the nano/microparticles in the manufacture of a medicament to treat that disorder.

### BRIEF DESCRIPTION OF THE DRAWINGS

The drawings are provided to illustrate some of the embodiments of the disclosure. It is envisioned that alternate configurations of the embodiments of the present disclosure maybe adopted without deviating from the disclosure as illustrated in these drawings.

FIG. 1 shows a schematic illustration of SparkCORAL™ microsphere/nanosphere preparation.

FIG. 2 shows a schematic illustration of a polycaprolactone (PCL) coating process on chitosan microspheres/nanospheres.

FIG. 3 shows SEM morphology of SparkCORAL™ microspheres (a) before release of BSA (b) and after release of BSA.

FIG. 4 shows a Fourier transform infra red (FTIR) spectrum of (a) polycaprolactone (PCL); (b) Crosslinked chitosan microspheres; and (c) BSA loaded chitosan microspheres.

FIG. 5 shows a FTIR spectrum of (a) polycaprolactone (PCL) (b) BSA loaded chitosan microspheres.

FIG. 6 shows a thermogravimetric analysis curves of (a) chitosan (b) chitosan microspheres (c) BSA loaded chitosan microspheres.

FIG. 7 shows thermogravimetric analysis curves of (a) polycaprolactone (PCL) (b) BSA loaded SparkCORAL™ microspheres.

FIG. 8 is an image of a gel showing SDS PAGE analysis of BSA, Lane 1: BSA before entrapment in SparkCORAL™ microspheres; Lane 2: BSA after release from SparkCORAL™ microspheres; Lane 3: Molecular weight ladder.

FIG. 9 shows a schematic illustration of incorporation of a drug or a therapeutic agent into chitosan microspheres/nanospheres.

FIG. 10 shows in vitro release data of BSA from chitosan microspheres demonstrating the effect of coating concentrations.

### DETAILED DESCRIPTION OF THE INVENTION

### I. Overview

One aspect of the invention provides a method of making delivery vehicles suitable for oral delivery of a therapeutic or diagnostic agent to a mammal, the method comprising (a) providing polymeric microparticles or nanoparticles comprising a therapeutic or diagnostic agent interspersed therein; (b) coating the polymeric particles, sequentially, with (i) a hydrophobic agent; and (ii) a hydrophilic agent; and optionally (c) encapsulating the coated polymeric particles in a gel capsule. In certain embodiments, the polymeric microparticles or nanoparticles comprise gelatin, alginate, chitosan, modified chitosan, polyamino acids, polypeptides, polycaprolactone/PEG mixture, dextran sulfate, dermatan sulfate, chondroitin sulfate, keratin sulfate, heparin sulfate, collagen, cellulose, elastin, or hyaluronic acid, or mixtures thereof. In certain embodiments, the polymeric microparticles or nanoparticles are chitosan particles, gelatin particles, alginate particles, protenoid particles or polycaprolactone/polyethylene-glycol particles.

In certain embodiments, the coating with the hydrophobic agent reduces lysosomal proteolysis of the polymeric particles or increases the period of release of the agent from the polymeric particle. In one preferred embodiment, the polymeric particles are microspheres or nanospheres. In certain preferred embodiments, the microspheres have a diameter of from about 2 μm to about 100 μm, and wherein the nanospheres have a diameter of from about 0.1 μm to about 0.8 μm. In certain preferred embodiments, the polymeric particles are coated by dip coating, spray coating, surface grafting or charge grafting. In certain preferred embodiments, the hydrophobic agent, the hydrophilic agent, or both, are polymers. In certain preferred embodiments, the hydrophobic polymer is selected from the group consisting of: (i) polycaprolactone (PCL); (ii) a combination of PCL and polyethylene glycol (PEG); and (iii) one or more of PLGA, PLA and PGA.

In certain preferred embodiments, the hydrophilic agent is a hydrophilic polymer. In certain preferred embodiments, the hydrophilic polymer is a cationic polymer. In certain preferred embodiments, the cationic polymer has amino groups, such as a concentration of amino groups in the cationic polymer of from about 7 meq/g to about 9 meq/g.

In certain preferred embodiments, the hydrophilic polymer is selected from chitosan, modified chitosan, derivatives and salts thereof. In certain preferred embodiments, the chitosan is modified to increase the density of positive charge. In certain preferred embodiments, the modified chitosan is aminated chitosan. In certain preferred embodiments, the gel capsule is coated with alginate. In certain preferred embodiments, the gel capsule releases the delivery vehicles in a pH-dependent manner, such as at an alkaline or neutral pH. In some embodiments, the method comprises treating the polymeric microparticles or nanoparticles with an aminating agent prior to the coating steps of (b). In certain preferred embodiments, the aminating agent is selected from ethylenediamine, spermine, jeffamine, 1-12 diaminododecane, 4,4″-diaminodicyclohexyl methane, 4,4″-diaminobibenzyl, cholaminochloride hydrochloride and cholestryamine.

In certain preferred embodiments, the therapeutic or diagnostic agent is selected from a polypeptide, nucleic acid, polysaccharide, lipid, glycoprotein, glycolipid, carbohydrate, small molecule, and combinations thereof. In certain preferred embodiments, the therapeutic agent is selected from an antibody, antibody fragment, enzyme, allergen, random copolymer, clotting factor, cholera protein, hepatitis protein, influenza protein, pneumonia protein, interferon, interleukin, chemokine, cytokine, growth hormone, antisense oligonucleotide, siRNA and shRNA. In certain preferred embodiments, the therapeutic agent is selected from human growth hormone (hGH), erythropoietin, insulin, calcitonin, collagen type II, tetanus toxoid, diphtheria toxoid, LHRH, basic fibroblast growth factor (bFGF), transforming growth factor, ciliary neurotrophic growth factor (CNTF) and epidermal growth factor (EGF).

In certain preferred embodiments, the diagnostic agent is a paramagnetic, radioactive or fluorogenic ion, preferably one that is detectable upon imaging. In certain preferred embodiments, the polymeric particles comprise glucose oxidase, catalase and insulin. In certain preferred embodiments, the polymeric particles do not comprise a protease inhibitor.

In certain preferred embodiments, at least 50% of the polymeric particles in the gel capsule are microspheres, nanospheres, or combinations thereof. In certain preferred embodiments, the agent is interspersed throughout the polymeric particles. In certain preferred embodiments, the polymeric microparticles or nanoparticles comprise a pH-sensitive hydrogel.

The invention also provides a delivery vehicle suitable for oral delivery of a therapeutic or diagnostic agent to a mammal prepared according to the methods provided herein. The invention also provides compositions suitable for oral administration comprising a plurality of delivery vehicles, preferably a plurality of coated polymeric particles, wherein a plurality of the polymeric particles are microspheres and a plurality of the polymeric particles are nanospheres, and optionally comprising a pharmaceutically-acceptable excipient.

The invention also provides a delivery vehicle suitable for oral delivery of a therapeutic or diagnostic agent to a mammal, the delivery vehicle comprising (a) a polymeric particle comprising the therapeutic or diagnostic agent interspersed therein; (b) a hydrophobic coating surrounding the polymeric particle; and (c) a hydrophilic coating surrounding the hydrophobic coating.

The invention also provides a multiple unit carrier system suitable for oral delivery of a therapeutic or diagnostic agent to a mammal, the system comprising (a) a gel capsule; and (b) a plurality of delivery vehicles contained within said gel capsule. In certain preferred embodiments, at least a portion of the plurality of delivery vehicles are microspheres and nanospheres. In certain preferred embodiments, the gel capsule is coated with alginate. In certain preferred embodiments, the gel capsule releases the delivery vehicles in a pH-dependent manner, such as at an alkaline or neutral pH.

The invention also provides a method of increasing the serum concentration of a therapeutic agent in an individual in need thereof, the method comprising orally administering to the subject a therapeutically-effective amount of a composition comprising the delivery vehicles or a multiple unit carrier system such as those provided herein. The invention also provides an use of delivery vehicles or a multiple unit carrier systems in the manufacture of a medicament to increase the concentration of a therapeutic or diagnostic agent in the serum of a mammal in need thereof. In certain preferred embodiments, the delivery vehicles comprise microspheres and nanospheres in a ratio of from about 1:100 to 100:1, more preferably from about 1:5 to about 5:1 by weight. In certain preferred embodiments, the microspheres and nanospheres comprise modified chitosan. In certain preferred embodiments, the agent is a polypeptide. In certain preferred embodiments, the agent is not a cytotoxic agent or an anticancer agent.

The invention also provides a delivery vehicle suitable for oral delivery of insulin to the bloodstream of a mammal, the delivery vehicle comprising (a) a chitosan sphere comprising insulin interspersed throughout; (b) a hydrophobic coating surrounding the chitosan sphere; and (c) a hydrophilic coating surrounding the hydrophobic coating. In certain preferred embodiments, the chitosan sphere is a pH-sensitive hydrogel.

The invention also provides a multiple unit carrier system for oral delivery of insulin to the bloodstream of a mammal, the delivery vehicle comprising (a) a plurality of chitosan spheres comprising insulin, catalase and glucose oxidase interspersed throughout; (b) a hydrophobic coating surrounding the chitosan spheres; (c) a hydrophilic coating surrounding the hydrophobic coating; and (d) a gel capsule containing the plurality of chitosan spheres. In certain preferred embodiments, the chitosan spheres further comprise catalase and glucose oxidase interspersed throughout. In certain preferred embodiments, the gel capsule releases the chitosan spheres in a pH-dependent manner. In certain preferred embodiments, the gel capsule is coated with alginate.

The invention also provides methods of preparing a plurality of nanospheres or microspheres comprising: (a) crosslinking chitosan with a dialdehyde; and (b) blocking residual or free aldehyde groups in the crosslinked chitosan; and optionally (c) further crosslinking the crosslinked chitosan of (b) with an agent that does not react with amino groups. In certain preferred embodiments, the dialdehyde is selected from glutaraldehyde, dialdehyde dextran, dialdehyde starch, alginate dialdehyde, chitosan dialdehyde, glucose dialdehyde, galactose dialdehyde, hyaluronic acid dialdehyde and heparin dialdehyde. In certain preferred embodiments, the dialdehyde is organic phase glutaraldehyde.

In certain preferred embodiments, the residual or free aldehyde groups are blocked with an amino acid. In certain preferred embodiments, the amino acid is selected from glycine, cysteine and histidine. In certain preferred embodiments, the residual or free aldehyde groups are blocked with an ethanolamine, an amino PEG or a diamino PEG. In certain preferred embodiments, the additional crosslinking agent in step (c) is epichlorohydrin, diisocyanate (e.g. hexamethylene diisocyanate) or blocked diisocyanate (e.g. a bisulfite adduct of 1,6-hexamethylene diisocyanate). In certain preferred embodiments, the chitosan is a modified chitosan, such as a cysteine-modified chitosan. In certain preferred embodiments, the chitosan is crosslinked with the dialdehyde in the presence of a therapeutic or a diagnostic agent, and preferably under conditions that allow the therapeutic or diagnostic agent to become interspersed throughout the microspheres or the nanospheres.

While the concepts of the present disclosure are illustrated and described in detail in the drawings and the description herein, such an illustration and description is to be considered as exemplary and not restrictive in character, it being understood that only illustrative embodiments are shown and described and that all changes and modifications that come within the spirit of the disclosure are desired to be protected.

### II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

Throughout this specification and claims, the word “comprise,” or variations such as “comprises” or “comprising,” will be understood to imply the inclusion of a stated integer or group of integers but not the exclusion of any other integer or group of integers.

The articles “a” and “an” are used herein to refer to one or to more than one

(i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably with the phrase, “such as but not limited to”.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “effective amount” as used herein is defined as an amount effective, at dosages and for periods of time necessary to achieve the desired result. The effective amount of a compound of the invention may vary according to factors such as the disease state, age, sex, and weight of the animal. Dosage regimens may be adjusted to provide the optimum therapeutic response. For example, several divided doses may be administered daily or the dose may be proportionally reduced as indicated by the exigencies of the therapeutic situation.

An “individual” as used herein refers to any vertebrate animal, preferably a mammal, and more preferably a human. Examples of individuals include humans, non-human primates, rodents, guinea pigs, rabbits, sheep, pigs, goats, cows, horses, dogs, cats, birds, and fish.

The term “polypeptide” encompasses native or artificial proteins, protein fragments, peptidomimetics and polypeptide analogs of a protein sequence. A polypeptide may be monomeric or polymeric. The term “peptide” refers to polypeptides having less than 30 amino acids in length.

The term “small molecule” refers to any molecule having a molecular weight of less than 2 kDa, preferably less than 1 kDa. Small molecules include both naturally-occurring and synthetic molecules.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant (e.g. chimeric and other derivatized) antibodies.

The term “polynucleotide” as referred to herein means a polymeric form of nucleotides of at least 10 bases in length, either ribonucleotides or deoxynucleotides or a modified form of either type of nucleotide. The term includes single and double stranded forms. Polynucleotides may comprise or consist of sequences that regulate gene expression, such as antisense polynucleotides, ribozymes, siRNA, shRNA and the like, and/or encode a gene product such as an mRNA or polypeptide product.

As used herein, the term “small interfering RNA” (“siRNA”) (also referred to in the art as “short interfering RNAs”) refers to an RNA (or RNA analog) comprising or consisting of between about 10-50 nucleotides (or nucleotide analogs) that are capable of directing or mediating RNA interference. Preferably, a siRNA comprises or consists of between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising or consisting of about 21 nucleotides (or nucleotide analogs), for example, 19, 20, 21 or 22 nucleotides. The term “long” siRNA refers to a siRNA comprising or consisting of about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “short hairpin RNA” or shRNA refers to an RNA molecule comprising at least two complementary portions hybridized or capable of hybridizing to form a double-stranded (duplex) structure sufficiently long to mediate RNAi (typically at least 19 base pairs in length), and at least one single-stranded portion, typically between approximately 1 and 10 nucleotides in length that forms a loop. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

The term “diagnostic” refers to assays that provide results which can be used by one skilled in the art, typically in combination with results from other assays, to determine if an individual is suffering from a disease or disorder of interest

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

As used herein, the term “chitosan” refers to a derivative of chitin, or poly-N-acetyl-D-glucosamine, in which the greater proportion of the N-acetyl groups have been removed through hydrolysis. Chitosan includes all derivatives of chitin, including all polyglucosamine and oligomers of glucosamine materials of different molecular weights, in which the greater proportion of the N-acetyl groups have been removed through hydrolysis. Preferably, chitosan has a positive charge.

As used herein, the term “microparticle” refers to particle having a volume in the range of about 0.5 to about 5×10⁵ μm³. Microparticles include particles capable of containing a therapeutic/diagnostic agent that is to be released within a mammalian body, including specialized forms such as microspheres and nanospheres, whether natural or artificial. Polymeric microparticles are microparticles formed (at least in part) from a crosslinkable polymer. Microparticles include microcapsules, microspheres, and microsponges.

As used herein, the term “nanoparticle” refers to a particle having a volume in the range of about 5×10⁴ to about 5×10⁸ nm³. Nanoparticles include particles capable of containing a therapeutic/diagnostic agent that is to be released within a mammalian body, including specialized forms such as nanospheres, whether natural or artificial. Polymeric nanoparticles are nanoparticles formed (at least in part) from a crosslinkable polymer. Nanoparticles include nanocapsules, nanospheres, and nanosponges.

As used herein, the term “microcapsule” refers to a microparticle wherein a polymeric wall encases a core consisting of an aqueous solution or suspension. In the case of microcapsules encapsulating hydrophobic active agents, the active agent may be contained in the core, in which case the core comprises an aqueous suspension of the active agent, or the active agent may be embedded in the polymeric wall itself, in which case the core comprises an aqueous solution or suspension substantially absent active agent.

As used herein, the term “microsponge” refers to a microparticle wherein an active agent is embedded within a polymeric matrix comprising an open-cell structure.

As used herein, the term “microsphere” refers to a substantially spherical particle in the size range of 1 to 100 micrometers in diameter. In one preferred embodiment, the microspheres are from about 2 to 50 micrometers in diameter. In another preferred embodiment, the microspheres are greater than 1 micrometer in diameter.

As used herein, the term “nanosphere” refers to a substantially spherical particle in the size range of 50 to 1000 nanometers in diameter. In one preferred embodiment, the nanospheres are from about 100 to 800 nanometers in diameter. In another preferred embodiment, the nanospheres are less than 1 μm in diameter. In another preferred embodiment, the nanospheres are less than 0.5 μm in diameter. In another preferred embodiment, the nanospheres are from about 50 nm to about 450 nm in diameter.

As used herein, the term “mucosal membrane” refers to any mucosal membrane in a mammalian body, e.g. human body. Mucosal membranes include, e.g., those of the buccal cavity, rectum, vagina, lungs, eye, colon, small intestine, stomach and nasal cavity.

As used herein, the terms “stimulus-sensitive hydrogels” are hydrogels that undergo a volume change in response to at least one stimulus (e.g., a chemical molecule or compound or a change on physical or chemical conditions such as pH, temperature, pressure, charge or ionic concentration). In the art, the term “stimuli-responsive”or “stimuli-sensitive” is used interchangeably to refer to hydrogels that similarly undergo a volume change in response to at least one stimulus.

### III. Synthesis/Modification/Loading of the Polymeric Particles

The invention provides improved methods for the synthesis of polymeric particles suitable for the transmucosal delivery of a therapeutic or diagnostic agent to the serum of a mammal. In preferred embodiments, the polymeric particles are microparticles or nanoparticles. One specific aspect of the invention provides methods of manufacturing, and compositions of, modified chitosan nanospheres and microspheres as carrier systems for oral delivery of therapeutic and diagnostic e.g. macromolecules. Compositions comprising modified chitosan nanospheres and microspheres may be used as carrier systems to deliver the agent in an active form to a mucosal membrane, such as an intestinal mucosal membrane.

One aspect of the invention provides a method of preparing a plurality of nanospheres or microspheres comprising (a) crosslinking chitosan with a dialdehyde; and (b) blocking residual or free aldehyde groups in the crosslinked chitosan. In specific embodiments, chitosan is modified by copolymerizing and/or blending with biocompatible synthetic cousins (chitosan derivatives).

In one embodiment, the chitosan has a high density of positive charge. In other embodiments, chitosan derivatives or salts (e.g: nitrate, phosphate, sulfate, hydrochloride, glutamate, lactate or acetate salts) of chitosan are used. Suitable chitosan derivatives include ester, ether, acyl and alkyl groups. Acyl and alkyl derivatives may be formed by bonding with the OH groups of chitosan while keeping intact its NH₂ groups. Other modified chitosans include conjugates of polyethylene glycol, polycaprolactone and graft copolymers with functional monomers/macromonomers. U.S. Pat. No. 6,730,735 describes methods for the modification of chitosan with PEG. Other suitable modifications can be performed to incorporate one or more types of functional groups in chitosan nano/microspheres.

In certain embodiments, functional groups are introduced to the chitosan to make it stimuli responsive or to increase its stimuli responsiveness. In one preferred embodiment, the chitosan is a modified chitosan, such as a cysteine-modified chitosan. Exemplary chitosan salts include nitrate, phosphate, sulphate, hydrochloride, glutamate, lactate or acetate salts. Low and medium viscosity chitosans (for example CL113, G210 and CL110) suitable for use in the methods described herein may be obtained from various sources, including Pronova Biopolymer, Ltd., UK; Seigagaku America Inc., Maryland, USA; Meron (India) Pvt, Ltd., India; Vanson Ltd, Virginia, USA; and AMS Biotechnology Ltd., UK. Suitable derivatives also include those which are disclosed in Roberts, Chitin Chemistry, MacMillan Press Ltd., London (1992) (incorporated herein by reference).

In addition, the chitosan may comprise chitosans of different molecular weights. Chitosans. In certain embodiments, the chitosan or chitosan derivative or salt used in the methods described herein preferably has a molecular weight of 4,000 Dalton or more, preferably in the range 25,000 to 2,000,000 Dalton, and most preferably about 50,000 to 300,000 Daltons. Chitosans of different low molecular weights can be prepared by enzymatic degradation of chitosan using chitosanase or by the addition of nitrous acid. Both procedures are well known to those skilled in the art and are described in recent publications (Li et al. (1995) Plant Physiol. Biochem. 33, 599-603; Allan and Peyron, (1995) Carbohydrate Research 277, 257-272; Damard and Cartier, (1989) Int. J. Biol. Macromol. 11, 297-302).

In certain embodiments, the chitosan is water-soluble and may be produced from chitin by deacetylation to a degree of greater than 40%, preferably between 50% and 98%, and more preferably between 70% and 90%. Particular deacetylated chitosans which may be used include the “Sea Cure®” series of chitosan glutamates available from Protan Biopolymer A/S, Drammen, Norway.

In certain embodiments, the crosslinking of chitosan with a dialdehyde is performed in a chitosan solution substantially free of other polymers. In some embodiments, the chitosan solution is substantially free of other biodegradable polymers. In some embodiments, the chitosan solution contains less than 50%, 40%, 30%, 20%, 15%, 10%, 5%, 2%, 1%, 0.5%, 0.2%, 0.1%, 0.05%, 0.02%, 0.01% w/v of other polymers or biodegradable polymers.

In certain preferred embodiments, the dialdehyde is selected from glutaraldehyde, dialdehyde dextran, dialdehyde starch, alginate dialdehyde, chitosan dialdehyde, glucose dialdehyde, galactose dialdehyde, hyaluronic acid dialdehyde and heparin dialdehyde. In one preferred embodiment, the dialdehyde is glutaraldehyde. In a more preferred embodiment, the glutaraldehyde is organic phase glutaraldehyde.

Production of chitosan nano/microspheres with high surface concentrations of aldehyde functionality may be achieved using organic phase glutaraldehyde. This facilitates a variety of chemical modifications such as coupling of amino acids, amino alcohols, proteins and other polyamino compounds to the nano/microspheres. Examples 1 and 2 exemplify the synthesis of microspheres and nanospheres, respectively, using organic phase glutaraldehyde.

In another embodiment, the method of preparing a plurality of nanospheres or microspheres further comprises blocking residual or free aldehyde groups in the crosslinked chitosan with an amino acid. Exemplary amino acids include glycine, cysteine and histidine. Hydrophobic amino acids may be used to increase the hydrophobicity of the chitosan. Alternatively or in combination, residual or free aldehyde groups may be blocked with an ethanolamine, an amino PEG or a diamino PEG.

The invention also provides a method of preparing a plurality of nanospheres or microspheres comprising (a) crosslinking chitosan with a dialdehyde; (b) blocking residual or free aldehyde groups in the crosslinked chitosan; and (c) further crosslinking the crosslinking chitosan of (b) with an agent that does not react with amino groups. Preferred agents for this step (c) of further crosslinking include epichlorohydrin, diisocyanate or blocked diisocyanate, such as hexamethylene diisocyanate or a bisulfite adduct of 1,6-hexamethylene diisocyanate.

In other embodiments, the therapeutic or diagnostic agents are incorporated into the nano/microspheres during their production or absorbed into the spheres after their production. Example 3 exemplifies both types of incorporation strategies for the incorporation of the polypeptide bovine serum albumin (BSA) into chitosan microspheres, while Example 15 describes the loading of nanospheres. Example 8 shows a method to quantify protein loading into microspheres. Accordingly, the invention provides a drug delivery composition in a form suitable for the administration to a mucosa, such as that of the intestine, comprising a therapeutic agent and nano/microspheres made from a mixture of various molecular weight chitosans or modified chitosans, wherein the agent is either incorporated into the nano/microspheres during production or is absorbed into the preformed nano/microspheres.

In certain preferred embodiments, the chitosan is crosslinked in the presence of a polymeric dispersing agent. In one preferred embodiment, the dispersing agent is poly methyl methacrylate (PMMA), such as from about 5% to about 20% PMMA. Various other polymeric dispersing agents, such as but not limited to, polaxymers, cellulose acetate butyrate, cellulose acetate phthalate, polycarbonate, polyurethane and the like can also be used in place of or in addition to PMMA in the preparation of the spheres.

Stirring time, power of stirring, dispersion time, concentration of chitosan and concentration of the dispersant polymers are factors that may also be adjusted by one skilled in the art to generate nanospheres of a desired size. For example, a while a 10% PMMA solution may be used to obtain chitosan microspheres with a particle size range of 1-10 micrometers (average of 6 micrometers) in diameter, lower concentrations may be used to obtain particles of smaller diameter.

Chitosan concentrations may also be increased to obtain larger particles. For instance, in a 10% PMMA solution at a stirring rate of 8000 rpm, microspheres in the size range of 1-9 micrometers may be obtained using 4% chitosan.

The stirring speed of the homogenizer also may be selected by one skilled in the art to generate chitosan microspheres of the desired size. In certain embodiments, the solution of polymer, such as chitosan, and dialdehyde is stirred during the crosslinking reaction in the range of about 5000 rpm to about 20,000 rpm. For example, using a 10% PMMA, 3.5% chitosan and glutaraldehyde (toluene saturated) 7.5 ml solution, stirring at 3,000 rpm or below results in aggregates forming a gel matrix. By stirring at 5000 rpm, microspheres in the range of 10-30 micrometer are generated. By stirring at speeds between 5000 to 7000 rpm, 8 to 15 micrometer chitosan microspheres are formed. By increasing the homogenizer stirring speed to 8000 rpm, chitosan micro spheres ranging in size from 1 to 10 micrometers are obtained.

In some embodiments, the chitosan nano/microspheres may be derivatized using functional reagents. Derivatization of chitosan nano/microspheres with aminating agents such as ethylenediamine, spermine, jeffamine, 1-12-diaminododecane, 4,4″-diaminodicyclohexyl methane, 4,4″-diaminobibenzyl, cholaminochloride hydrochloride, cholestryamine, acylating agents, alkylating agents, esterification reagents, and reacting with an acid or base to provide salt/base derivative such as nitrate, phosphate, sulfate, hydrochloride, glutamate, lactate or acetate salts, may be performed either by directly derivatizing chitosan using standard protocols prior to nano/microspheres formation or by derivatizing the nano/microspheres after formation. In certain embodiments, modified chitosans or chitosan conjugates are prepared by conjugating chitosan with polyethylene glycol, polycaprolactone and graft copolymers with functional monomers/macromonomers using standard protocols.

Another aspect of the invention provides methods of modifying polymeric nano/microparticles to generate delivery vehicles for improved transmucosal delivery of therapeutic/diagnostic agents to a mammal. One aspect provides methods of sequentially coating polymeric particles with hydrophobic and hydrophilic agents, preferably polymers. The polymeric nano/microparticles coated according to the methods provided herein exhibit at least one of the following properties: (a) greater resistance to stomach proteases; (b) enhanced mucoadhesivity; (c) enhanced penetration of a mucosal layer; (d) enhanced trans-cellular or paracellular transport across epithelial cells that are adjacent to a mucosal layer, such as intestinal epithelial cells; (e) decreased phagocytosis by M-cells in Peyer's patches; (f) greater resistance to degradation by lysosomal proteases; and (g) extended release of the therapeutic/diagnostic agent, such as into the bloodstream. In one preferred embodiment, at least one of the coatings reduces lysosomal proteolysis of the polymeric particles.

Some aspects of the invention for modifying polymeric nano/microparticles comprise (a) providing polymeric particles comprising a therapeutic or diagnostic agent interspersed therein; (b) coating the polymeric particles, sequentially, with (i) a hydrophobic agent; and (ii) a hydrophilic agent; and optionally, (c) encapsulating the coated polymeric particles in a gel capsule, which itself may be optionally coated. A related aspect of the invention provides a method of making delivery vehicles suitable for transmucosal delivery of a therapeutic or diagnostic agent to a mammal, the method comprising (a) providing polymeric microparticles or nanoparticles comprising a therapeutic or diagnostic agent interspersed therein; (b) coating the polymeric particles, sequentially, with (i) a hydrophobic agent; and (ii) a hydrophilic agent; and optionally (c) encapsulating the coated polymeric particles in a gel capsule, which itself may be optionally coated.

In some embodiments, step (i), step (ii), or both, are repeated to generate a double coat of a hydrophobic agent, a hydrophilic agent, or of both. In some embodiments, the second hydrophobic agent is the same as the first, while in others it is a different one, which may include a derivative of the first or a different agent altogether. Similarly, in some embodiments, the second hydrophilic agent is the same as the first, while in others it is a different one, which may include a derivative of the first or a different agent altogether. Example 4 illustrates the application of a double coating of a hydrophobic polymer, polycaprolactone (PCL), to chitosan microspheres while Example 16 that of nanospheres.

The coating steps may be achieved using any methods known in the art for coating particles, including but not limited, to dip coating, spray coating, surface grafting or charge grafting.

In certain embodiments, the polymeric particles comprise gelatin, alginate, chitosan, modified chitosan, polyamino acids, polypeptides, polycaprolactone/PEG mixture, dextran sulfate, dermatan sulfate, chondroitin sulfate, keratin sulfate, heparin sulfate, collagen, cellulose, elastin, or hyaluronic acid, or mixtures thereof. In other embodiments, the polymeric particles comprise poly(lactic acid)s, poly(glycolic acid)s, polyanhydrides, polyorthoesters, polycaprolactones, polyphosphazenes, polyhydroxybutyrates, polyamides, blends and copolymers thereof.

In certain preferred embodiments, the polymeric microparticles or nanoparticles are chitosan particles, gelatin particles, alginate particles, protenoid particles or polycaprolactone/polyethylene-glycol spheres. Methods for the preparation of protein-based microspheres are described in U.S. Pat. Nos. 5,271,961, 5,069,936 and 4,107,288. In some embodiments, the polymeric particles comprise a combination of two or more polymers. For example, U.S. Pat. No. 6,465,626 describes microspheres comprising a mixture of chitosan and gelatin A and methods for their preparation. In preferred embodiments, the polymer content of the polymeric particles is at least 10, 20, 30, 40, 50, 60, 70, 80, 90, 95, 98, 99 or 100% chitosan.

In other embodiments, the polymeric microparticles are stimuli-sensitive hydrogels. A number of stimulus-sensitive hydrogel compositions may be used in the methods or compositions of the present invention, including hydrogels that respond to physical changes, such as temperature or pressure changes; to chemical changes, such as a pH or ionic strength change; to chemical compounds, including metal ions (for example, Pb(II), Fe(II); Fe(III), Cr(III), Mn(II), Mg(II), Cu(II), Pb(II)), lactate, lactic acid, ethanol, ammonia, glycerol, and glucose; and to biological agents, such as toxins, proteins, pathogens, hormones, nucleic acids, antibodies, viruses, and peptides.

In one preferred embodiment, the hydrogels are sensitive to pH. One exemplary pH sensitive hydrogel includes 2-(dimethylamino)ethyl methacrylate as a cross-linker, 2-hydroxyethyl methacrylate as a comonomer, ethylene glycol dimethyacrylate, and 2,2-dimethoxy-2-phenyl acetophenone as a photoinitiator. Another example of a pH sensitive hydrogel is poly(N-isopropyl acrylamide-co-methacrylic acid) (NIPA/MAA), which swells with increasing pH. Another pH sensitive hydrogel is cationic chitosan.

In certain specific embodiments of the invention, a pH-sensitive hydrogel used in the preparation of a nano/microparticle may comprise the following basic monomer units (in the context of the polymer): aminoethyl methacrylate; N,N-dimethylaminoethyl methacrylate (Siegel and Firestone, 1988, Macromolecules 21:3254; Siegel, 1990, in “Pulsed and Self-regulated Drug Delivery”, Kost, ed., CRC Press, Boca Raton, Fla., pp. 129-157); N,N-diethylaminoethyl methacrylate (Kopecek et al., 1971, J. Polym. Sci. 9:2801; Vacik and Kopecek, 1975, J. Appl. Polym. Sci. 19:3029; Ishihara et al., 1984, Polymer J. 8:625); a moiety comprising imidazole; a moiety comprising an imidazole derivative; vinylpyridine; and vinylbenzyl trimethylammonium chloride (Michaels et al., 1961, J. Phys. Chem. 65:1765; Michaels, 1965, Ind. Eng. Chem. 57:32).

In other preferred embodiments of the invention, a pH-sensitive hydrogel used in the preparation of a nano/microparticle may comprise the following acidic monomer units (in the context of the polymer): acrylic acid or methacrylic acid (Ricka and Tanaka, 1984, Macromolecules 17:2916; Kopecek et al., 1971, J. Polym. Sci. 9:2801; Kou et al., 1988, Pharm. Res. 5:592; Vacik and Kopecek, 1975, J. Appl. Polym. Sci. 19:3029); alkyl methacrylate esters, and particularly methylmethacrylate ester (Siegel and Firestone, 1988, Macromolecules 21:3254-3259); sodium styrenesulfonate (Michaels et al., 1961, J. Phys. Chem. 65:1765; Michaels, 1965, Ind. Eng. Chem. 57:32); and sulfoxyethyl methacrylate (Kudela et al., 1980, J. Membrane Sci. 6:123).

In still other preferred embodiments of the invention, a pH-sensitive hydrogel used in the preparation of a nano/microparticle may comprise a polyelectrolyte complex formed from an anionic polymer and a cationic polymer, where, at pH less than 7, cations are the predominant ionic species, and the polymer pairs may be as follows: poly(vinylbenzyl trimethylammonium chloride) and sodium poly(styrene sulfonate) (Ratner and Hoffman, 1976, in “Hydrogels for Medical and Related Applications”, Andrade, ed., ACS Symposium Series 31, American Chemical Society, Washington, D.C., pp. 1-36); each of which is incorporated herein by reference; and copolymers based on n-alkyl methacrylate esters and (dimethylamino)ethyl methacrylate (Siegel and Firestone, 1988, Macromolecules 21:3254-3259).

In certain embodiments, the polymeric particles to be coated are microspheres or nanospheres, preferably nanospheres. In preferred embodiments, the polymeric particles have a diameter, or a length if the particle is not substantially spherical, of less than 25, 20, 15, 12, 10, 9, 8, 7, 6, 5, 4, 3, 2, 1, 0.9, 0.7, 0.6, 0.5, 0.4, 0.3, 0.2 or 0.1 μm. In certain embodiments, the microspheres to be coated have a diameter from about 2 μm to about 100 μm, and the nanospheres have a diameter of from about 0.05 μm to about 0.8 μm, more preferably between 0.1 μm and 0.8 μm. In some embodiments, the nanospheres are less than 0.5 μm in diameter.

The particles to be coated may be prepared using any of the methods described herein for the preparation of nano/microparticles or of nano/microspheres. Alternatively, any method known in the art for the preparation of these particles may be used, including but not limited to, by solvent evaporation, phase separation, spray drying, emulsification or precipitation.

Methods of forming microspheres by solvent evaporation are described in U.S. Pat. No. 4,389,330 and in Canadian Patent Nos. CA 2,100,925 and CA 2,099,941. Solvent evaporation may be useful for the encapsulation of lipophilic substances such as steroids and nitrosoureas.

Another technique which can be used to form microparticles is phase separation, which involves the formation of a water-in-oil emulsion or oil in water emulsion (see, e.g., U.S. Pat. Nos. 4,675,189, 4,675,800, 4,835,139, 4,732,763, and 4,897,268). The process of phase separation usually employs an emulsion or a suspension of the drug particles in a solution of a high molecular weight polymer and an organic polymer solvent. A non-solvent is then added to the suspension or emulsion, causing the polymer to separate from solution and to encapsulate the suspended drug particles or droplets containing them. The resulting microparticles (which are still swollen with solvent) are then normally hardened by a further addition of a non-solvent or by some other process which strengthens and improves the properties of the microparticles.

An emulsion-based example of a process to form microparticles described in U.S. Pat. No. 3,523,906 may be used to obtain polymeric particles for use in the methods of the invention. In this process, a material to be encapsulated is emulsified in a solution of a polymeric material in a solvent which is immiscible with water and then the emulsion is emulsified in an aqueous solution containing a hydrophilic colloid. Solvent removal from the microcapsules is then accomplished in a single step by evaporation and the product is obtained.

A spray-drying process for producing polymeric particles in which water-soluble peptides and proteins can be incorporated for use in the methods of the invention is described in European Patent No. 0315875. In this process, an aqueous peptide or protein solution is emulsified in an organic polymer solution and this emulsion is then spray-dried. Examples of other spray drying processes are disclosed, e.g. in U.S. Pat. Nos. 5,648,096, 5,723,269, and 5,622,657.

Additional processes for producing polymeric particles suitable for use in the methods of the invention are disclosed in U.S. Pat. Nos. 6,291,013, 5,792,477, 5,643,605, 5,922,357, 6,309,569, 6,224,794, U.S. Pat. Pub Nos 2002/0192294 and 2003/0183962, and in PCT publications WO 99/59548 and WO 01/28591. Similarly, U.S. Pat. No. 5,863,554 shows, for example, the formation of starch microspheres by emulsion and by coacervation, albumin microspheres by emulsification and by coacervation, gelatin microspheres by emulsion, gelatin microspheres by coacervation and chitosan microspheres by emulsion, any of which may be used in conjunction with the coating methods described herein.

In certain preferred embodiments of the methods described herein for the coating of polymeric particles, such as the coating of nano/microspheres, the hydrophobic agent, the hydrophilic agent, or both, are polymers. In some embodiments, the hydrophobic polymer is (i) polycaprolactone (PCL); (ii) a combination of PCL and polyethylene glycol (PEG); or (iii) one or more of poly(lactic acid-glycolic acid) (PLGA), poly(lactic acid) (PLA) and poly(glycolic acid) (PGA). In one preferred embodiment, the hydrophobic polymer is PCL, whose biocompatibility and biodegradability is known.

The hydrophobic polymer may used in combination with other polymers to manipulate the rate of release of drugs from nano/microspheres. As shown in Example 9, PCL coating of chitosan microspheres extended the protein release period of the microspheres. The hydrophobic polymer coating, such as a PCL coating, may be useful to promote the uptake by intestinal cells.

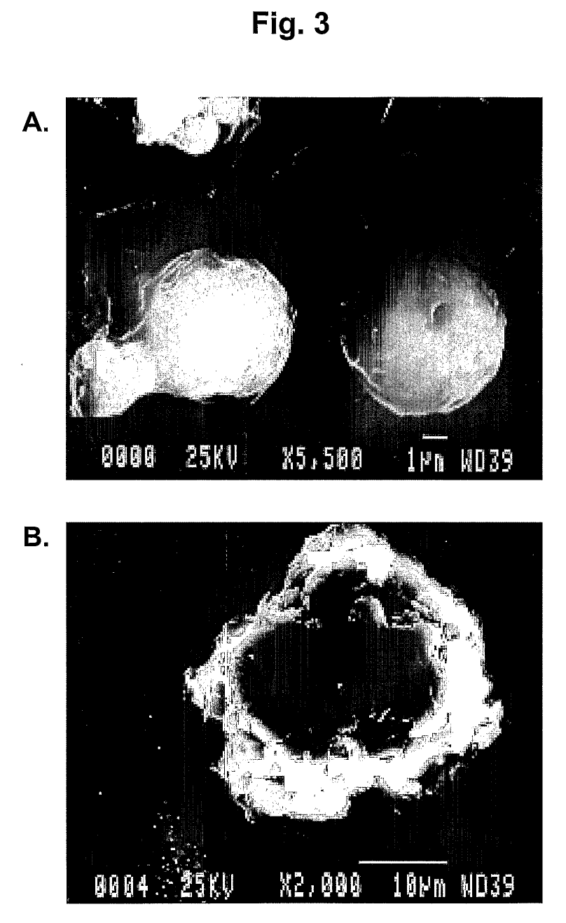

In one specific exemplary embodiment, SparkCORAL™ nano/microspheres are coated with polycaprolactone (PCL) to impart hydrophobicity followed by a modified chitosan coating to impart hydrophilicity. Examples 5-7 and 17 (and FIGS. 3-7) exemplify the characterization of PCL-coated and uncoated nano/microspheres. Without being bound by a particular mechanism, it is believed that the chitosan coating, among other things, protects the particles from aggregation and lysozymic degradation, and the PCL imparts the required hydrophobicity for the particles to travel through the transcellular pathway of intestinal cells. Examples 4 and 16 exemplify the coating of microspheres and nanospheres with PCL, respectively.

In some embodiments, the hydrophobic polymer is combined with polyethylene glycol (PEG) for coating of the particles. In a specific embodiment, PCL and PEG are used to coat the particles. Since PCL biodegradation is very slow and the rate of release is prolonged for a very long time, combination with PEG allows tuning of release characteristics due to increased biodegradability of the coated film and increased biocompatibility, increased circulation time, prevention of attacks from the body's immune system, resistance to protein absorption and internalization efficiency. In other embodiments, the hydrophobic agent is a fatty acid, such as palmitoyl, or a hydrophobic amino acid.

In certain preferred embodiments, the hydrophilic agent is a hydrophilic polymer, preferably a cationic polymer. In some embodiments, the cationic polymer has amino groups. In certain specific embodiments, the concentration of amino groups in the cationic polymer ranges from about 5 meq/g to about 11 meq/g, more preferably from about 7 meq/g to about 9 meq/g, or about 8 meq/g. In some embodiments, the hydrophilic polymer is chitosan, modified chitosan, derivatives or salts thereof. Modified chitosans that may be used to coat the particles include, but are not limited to, chitosan modified to increase the density of positive charge. An exemplary modified chitosans is aminated chitosan. Other suitable chitosan derivatives also include ester, ether, acyl and alkyl groups. Acyl and alkyl derivatives are formed by bonding with the OH groups of chitosan keeping intact its NH₂ groups. In other embodiments, the hydrophilic polymer is an anionic chitosan. Anionic chitosan is described, for example, in Kochkina et al., (2000) Mikrobiologiia; 69(2): 257-60.

In another embodiment of the methods for making coated-particle delivery vehicles, the coated nano/micro particles are further coated with third hydrophilic agent. In one preferred embodiment, the hydrophilic agent is a pH-dependent polymer such as alginate.

In some certain embodiments, the methods for making delivery vehicles further comprises treating the polymeric microparticles or nanoparticles with an aminating agent prior to step (b) of coating the particles. Aminating agents include, but are not limited to, ethylenediamine, spermine, jeffamine, 1,12-diaminododecane, 4,4″-diaminodicyclohexyl methane, 4,4″-diaminobibenzyl, cholaminochloride hydrochloride and cholestryamine. Amination of the particles may be used to increase their net positive charge, which may aid in the loading of anionic drugs. Example 14 exemplifies a methods for measuring the positive charge on aminated chitosan particles. Furthermore, amination may be used to increase the pH dependent swelling of the particles, and in particular those particles that contain chitosan.

In certain embodiments of the methods for making coated-particle delivery vehicles, the coated nano/microparticles are encapsulated into a gel (such as a gel capsule) or other delivery device, including the multiple unit carrier system described in section IV of the instant specification. Preferably, the delivery device or gel is a stimuli responsive delivery device or gel, such that the coated nano/microparticles are released in response to a stimulus or a change in the external environment of the device/gel. Stimuli which may promote the release of the particles include, for example, temperature, pH, ionic strength, solvent, pressure, stress, light intensity, electric field, magnetic field and gelling agents.

In one embodiment, the gel capsule, e.g. a gelatin capsule, is coated with alginate. In another embodiment, the gel capsule releases the delivery vehicles in a pH-dependent manner. The release may occur at alkaline, acidic, or nearly physiological pH. In certain preferred embodiments, the gel capsule releases the delivery vehicles at an alkaline or neutral pH.

In the methods described herein, the polymeric particles comprising a therapeutic or diagnostic agent are not limited to any particular therapeutic or diagnostic agent. Section VI of the instant specification describes therapeutic or diagnostic agents that may be used in conjuction with the methods described herein. In certain preferred embodiments, the therapeutic or diagnostic agents are polypeptides, polynucleotides, nucleoproteins, polysaccharides, glycoproteins, lipoproteins, polypeptides, polysaccharides, lipids, glycolipids, carbohydrates or small molecules.

In certain embodiments, the therapeutic or diagnostic agent is an antibody, antibody fragment, enzyme, allergen, random copolymer, clotting factor, cholera protein, hepatitis protein, influenza protein, pneumonia protein, interferon, interleukin, chemokine, cytokine, growth hormone, antisense oligonucleotide, siRNA or shRNA. In certain preferred embodiments, the therapeutic or diagnostic agent is human growth hormone (hGH), erythropoietin, insulin, calcitonin, collagen type II, tetanus toxoid, diphtheria toxoid, LHRH, basic fibroblast growth factor (bFGF), transforming growth factor, ciliary neurotrophic growth factor (CNTF) or epidermal growth factor (EGF).

In other embodiments, the therapeutic agent is selected from steroids, analgesics, local anesthetics, antibiotic agents, chemotherapeutic agents, immunosuppressive agents, anti-inflammatory agents, antiproliferative agents, antimitotic agents, angiogenic agents, antipsychotic agents, central nervous system (CNS) agents; anticoagulants, fibrinolytic agents, growth factors, antibodies, ocular drugs, and metabolites, analogs, derivatives, fragments, and purified, isolated, recombinant and chemically synthesized versions of these species.

In one preferred embodiment, the polymeric particles comprise insulin. In another preferred embodiment, they comprise insulin, glucose oxidase and catalase. In one such embodiment, the polymeric particle is a glucose-sensitive insulin-releasing polymeric particle.

A related aspect of the invention provides delivery vehicles, gels, gel capsules, delivery devices or multiple unit carriers systems, prepared according to any of the methods described herein. The invention further comprises compositions comprising (i) any of the delivery vehicles, gels, gel capsules, delivery devices or multiple unit carriers systems described herein, or manufactured according to the methods described herein; and (ii) a pharmaceutically acceptable excipient. Pharmaceutically acceptable excipients include, but are not limited to, binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); and wetting agents (e.g., sodium lauryl sulphate).

In preferred embodiments, the compositions comprise both microspheres and nanospheres. In certain embodiments, the weight/weight ratio of nanospheres to microspheres is at least about 0.001, 0.005, 0.01, 0.05, 0.1, 0.2, 0.3, 0.4, 0.5, 0.6, 0.7, 0.8, 0.9, 1, 1.5, 2, 5, 10, 20, 50, 100, 200, 500 or 1000. In one embodiment wherein the compositions comprise both nanospheres of microspheres, the microspheres are greater than 1 μm in diameter and the nanospheres are less than 1 μm in diameter. In another embodiment, the composition comprises microspheres and nanospheres, wherein the difference between the average size of the microspheres and the average size of the nanospheres differs by at least 0.1, 0.2, 0.3, 0.5, 1, 1.5, 2, 3, 4, 5, 6, 7, 8, 9 10, 15 or 20 μm. In another embodiment, the composition comprises microspheres and nanospheres, wherein the composite distribution of their size in the composition is a mixture-Gaussian distribution comprising two Gaussian distributions each having its own mean, wherein the two means differ from each other by least 0.1, 0.2, 0.3, 0.5, 1, 1.5, 2, 3, 4, 5, 6, 7, 8, 9 10, 15 or 20 m.

### IV. Multiple Unit Carrier System

In addition to providing polymeric particles/delivery vehicles and methods of making them, the invention also provides multiple unit carrier systems suitable for transmucosal delivery of a therapeutic or diagnostic agent to a mammal. One specific aspect of the invention provides a multiple unit carrier system suitable for transmucosal delivery of a therapeutic or diagnostic agent to a mammal, the system comprising (a) a gel capsule; and (b) a plurality of delivery vehicles contained within said gel capsule. In a preferred embodiment, the multiple unit carrier system is suitable for oral administration. In one embodiment, the gel capsule is a gelatin capsule. In one embodiment, at least a portion of the plurality of delivery vehicles are microspheres and nanospheres. In another preferred embodiment, the gel capsule releases the delivery vehicles in a pH-dependent manner.

In another embodiment, the gel capsule is gelatin coated with alginate. In an exemplary embodiment, a gel capsule may include a single or double coat of 5-30%, or more preferably 15-25%, or more preferably about 20% w/v of a pH sensitive sodium alginate and a cross-linking agent such as calcium. Sources for the crosslinking calcium ions used in the formation of alginate gels include, for example, calcium carbonate, calcium sulfate, calcium chloride, calcium phosphate, calcium tartrate, calcium nitrate, and calcium hydroxide. Other acceptable crosslinkers may include lanthanum chloride, ferric chloride, cobaltous chloride, as generally are other compounds with multivalent cations, such as calcium (Ca++), copper (Cu++), barium (Ba++), strontium (Sr++) and the like. The gel capsule of the invention may be chemically evaluated for its in vivo gastrointestinal tract behavior. In a certain preferred embodiments, therapeutic agents are vaccines, insulin, hGH, erythropoietin, calcitonin, collagen Type II and growth factors.

In a preferred embodiment, the multiple unit carrier system comprises a combination of microspheres or nanospheres as carriers for the therapeutic agents. In certain embodiments, the weight/weight ratio of nanospheres to microspheres is at least about 0.001, 0.005, 0.01, 0.05, 0.1, 0.2, 0.3, 0.4, 0.5, 0.6, 0.7, 0.8, 0.9, 1, 1.5, 2, 5, 10, 20, 50, 100, 200, 500 or 1000. In one embodiment wherein the compositions comprise both nanospheres of microspheres, the microspheres are greater than 1 μm in diameter and the nanospheres are less than 1 μm. In another embodiment, the composition comprises microspheres and nanospheres, wherein the difference between the average size of the microspheres and the average size of the nanospheres differs by at least 0.1, 0.2, 0.3, 0.5, 1, 1.5, 2, 3, 4, 5, 6, 7, 8, 9 10, 15 or 20 μm. In another embodiment, the composition comprises microspheres and nanospheres, wherein the composite distribution of their size in the composition is a mixture-Gaussian distribution comprising two Gaussian distributions each having its own mean, wherein the two means differ from each other by least 0.1, 0.2, 0.3, 0.5, 1, 1.5, 2, 3, 4, 5, 6, 7, 8, 9 10, 15 or 20 μm.

Without intending to be bound by mechanism, the multiple unit carrier system of the invention allows the nano/microparticles to resist inactivation by digestive enzymes in the gastrointestinal (GI) system, mainly in the stomach, because of a gel capsule such as a gelatin gel capsule. This capsule, as a carrier, protects the protein from the harsh environment of the stomach before releasing the therapeutic agent into more favorable regions of the GI tract in a pH dependent manner, specifically the lower regions of the intestine.

A related aspect of the invention provides a method of delivering a plurality of nanospheres or microspheres includes the steps of: (a) obtaining nanospheres or microspheres crosslinked with organic phase glutaraldehyde comprising a suitable therapeutic agent; and (b) preparing a gelatin capsule device to transport the nanospheres or microspheres containing the therapeutic agent to deposit them selectively in a suitable tissue where a therapeutic action or drug absorption is desired.

Although multiple-unit carrier systems based on gel capsules are preferred for oral administration of the delivery vehicles of the invention, the invention also provides additional pharmaceutical compositions for the oral delivery of delivery vehicles, and in particular, of chitosan nano/microspheres. Such pharmaceutical compositions may take the form of, for example, tablets prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration of the delivery vehicles may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The invention also provides compositions, suitable for administration by inhalation, which comprise the polymeric particles or the delivery vehicles described herein. The delivery vehicles of the present invention, including nano/microspheres, may be conveniently delivered to an individual in the form of an aerosol spray presentation from pressurized packs or from a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The invention also provides compositions, suitable for administration through the oral mucosa, which comprise the polymeric particles or the delivery vehicles described herein. Oral transmucosal delivery refers to the delivery of a delivery vehicle across a mucous membrane in the oral cavity, pharyngeal cavity, or esophagus, and may be contrasted, for example, with traditional oral delivery, in which absorption of the drug occurs in the intestine. Accordingly, routes of administration in which the delivery vehicles is absorbed through the buccal, sublingual, gingival, pharyngeal, and/or esophageal mucosa are all encompassed within “oral transmucosal delivery,” as that term is used herein. For administration through the transmucosal mucosa, the delivery vehicles may be formulated, for example, into chewing gums (see U.S. Pat. No. 5,711,961) or buccal patches (see e.g. U.S. Pat. No. 5,298,256).

The invention also provides compositions, suitable for administration through the vaginal mucosa, which comprise the polymeric particles or the delivery vehicles described herein. The delivery vehicles of the invention may be formulated into a vaginal suppository, foam, cream, tablet, capsule, ointment, or gel.

In certain embodiments, pharmaceutical compositions comprising the delivery vehicles are formulated with permeants appropriate to the barrier to be permeated. Such penetrants are generally known in the art, and include, for example, for transmucosal administration bile salts and fusidic acid derivatives. In other embodiments, the pharmaceutical compositions comprising the delivery vehicles do not comprise permeants.

### V. Glucose-Sensitive Polymeric Particles

Another aspect of the invention provides glucose-sensitive polymeric particles (GSPP) and glucose-sensitive delivery devices (GSDD). The GSSPs or GSDDs of the invention are self-regulated insulin delivery systems that can administer increasing amounts of insulin in response to an increased glucose concentration in the blood. In some embodiments, they combine glucose oxidase with pH sensitive hydrogels to sense glucose and regulate insulin release from the hydrogels. Without intending to be bound by mechanism, the micro-environmental pH in the GSPPs becomes low, due to the production of gluconic acid. As the membrane swells, resulting in the ionization of the amino groups by the lower pH, insulin permeation through the membrane is enhanced. Thus insulin permeation through the membrane is strongly dependent upon the glucose concentration (ionization is the driving force for the swelling behavior).

In a preferred embodiment, a pH-dependent microgel, such as chitosan or aminated chitosan, is used in the preparation of a glucose sensitive hydrogel (GSH) because of its cationic charge and also for its mucoadhesivity, biodegradability and ability to enhance penetration of large molecules across mucosal surfaces. For insulin delivery, GSH responds to glucose concentration changes in the body almost immediately. A pH-sensitive hydrogel cannot react to changes in glucose concentration until the pH value inside the hydrogel changes. After the pH value inside the gel changes, mass transfer of water into or out of the gel occurs. The mass transfer of water is driven by osmotic pressure forces, which in turn are generated by ion-exchange processes and polymer conformational changes within the gel. The pH-sensitive hydrogels may have ionizable groups such as carboxylic acids, amino groups and sulfanilamide groups. Ionization of these groups generates strong osmotic pressure forces and swells the gel; deionization of these groups de-swells (shrinks) the gel. The time it takes a species to diffuse into the interior of the gel can be increased by decreasing the gel thickness and/or by increasing the gel porosity using techniques well-known to one skilled in the art.

The chitosan in the GSPP may be modified by amination because high amino content induces high sensitivity to pH changes. In addition, high cationic charge density imparts endosomal buffering capacity to suppress the endosomal enzyme activities and thereby protecting the therapeutic protein from degradation. Because crosslinking density also affects the response time, the degree of cross linking may be optimized to obtain the best reversible swelling pH.

Accordingly the invention provides a method of preparing a plurality of nanospheres or microspheres for use in a glucose responsive or a glucose stimuli sensitive drug delivery system comprising the steps of: preparing a plurality of nanospheres or microspheres; and immobilizing a plurality of glucose oxidase molecules in the nanospheres or microspheres. These steps may be performed sequentially or simultaneously. Examples 10 and 11 exemplify the loading of insulin into chitosan microspheres.

In a preferred embodiment, the method of preparing a plurality of nanospheres or microspheres for use in a glucose responsive or a glucose stimuli sensitive drug delivery system further includes a step of co-immobilizing the glucose oxidase with a plurality of catalase molecules. The method further includes immobilizing insulin. Preferred concentrations of glucose oxidase range from about 500 units/ml to 1500 units/ml and preferred concentrations of catalase range from about 100 units/ml to about 1000 units/ml.

A related aspect of the invention provides a delivery vehicle suitable for oral delivery of insulin to the bloodstream of a mammal, the delivery vehicle comprising (a) a chitosan sphere comprising insulin interspersed throughout; (b) a hydrophobic coating surrounding the chitosan sphere; and (c) a hydrophilic coating surrounding the hydrophobic coating. In a preferred embodiment, the chitosan sphere is a pH-sensitive hydrogel.

Another related aspect of the invention provides a multiple unit carrier system for oral delivery of insulin to the bloodstream of a mammal, the delivery vehicle comprising (a) a plurality of chitosan spheres comprising insulin, catalase and glucose oxidase interspersed throughout; (b) a hydrophobic coating surrounding the chitosan spheres; (c) a hydrophilic coating surrounding the hydrophobic coating; and (d) a gel capsule containing the plurality of chitosan spheres. In another embodiment, the gel capsule releases the chitosan spheres in a pH-dependent manner, preferably in the alkaline pH of the small intestine. In another preferred embodiment, the gel capsule is coated with alginate. Example 18 exemplifies the preparation of a capsule-based GSPP containing a cocktail on microspheres and nanospheres which release insulin.

### VI. Therapeutic/Diagnostic Agents

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics. Examples 3, 11 and 15 exemplify the loading of nano/microspheres with polypeptide therapeutic agents, while Example 12 exemplifies the loading of nucleic acids.

The therapeutic agents of the invention include small molecules, peptides, proteins, antigens, antibodies, antibody fragments, hormones, enzymes, vaccines, carbohydrates, lipids, liposomes, lipoproteins, enzyme inhibitors, cells, DNA, RNA, antisense oligonucleotides, genes, gene constructs such as plasmids, viral vectors, or a combination thereof. In some embodiments, the therapeutic agent is an isolated protein. In certain preferred embodiments, the therapeutic agent is insulin, hGH (human growth hormone), erythropoietin, calcitonin, tetanus toxoid, diphtheria toxoid, type II collagen.

For applications employing such materials as genes, gene constructs, vaccines and the like, the nano/microparticles are useful to enhance the delivery of the therapeutic agent into the mucosal tissue for enhanced therapeutic effect, for example in the presentation of antigen to the underlying lymphoid tissue, and/or transfection of the cells in the mucosal lining. The therapeutic agent can be used in combination with a pharmaceutically acceptable carrier/excipient.

The nano/microparticles and the delivery systems of the invention are particularly useful for oral administration of vaccines such as tetanus toxoid, diphtheria toxoid, cholera, hepatitis, influenza, pneumonia; proteins such as human growth hormone (hGH), erythropoietin, calcitonin, hormones such as insulin, growth hormones, LHRH; anti-cancer therapeutic macromolecules such as interferons, interleukins, growth factors such as basic fibroblast growth factor (bFGF), transforming growth factor, ciliary neutrophic growth factor (CNTF), epidermal growth factor (EGF) and polynucleotides such as antisense oligonucleotides and siRNA/shRNAs. Therefore, nano/microspheres disclosed are broadly useful as carriers of therapeutic peptides, proteins, nucleic acids and other macromolecules.

Further therapeutic agents include but are not limited to: antibiotics and antimicrobial agents, such as tetracycline hydrochloride, leucomycin, penicillin, penicillin derivatives, erythromycin, sulphathiazole and nitrofurazone; anti-migraine compounds, such as naratriptan, sumatriptan, alnitidan or other 5-HT1 agonists; vasoconstrictors, such as phenylephedrine hydrochloride, tetrahydrozoline hydrochloride, naphazolinie nitrate, oxymetazoline hydrochloride and tramazoline hydrochloride; cardiotonics, such as digitalis and digoxin; vasodilators, such as nitroglycerine and papaverine hydrochloride; bone metabolism controlling, agents, such as vitamin D and active vitamin D3; sex hormones; hypotensives; anti-tumour agents; steroidal anti-inflammatory agents, such as hydrocortisone, prednisone, fluticasone, prednisolone, triamcinolone, triamcinolone acetonide, dexamethasone, betamethasone, beclomethasone and beclomethasone dipropionate; non-steroidal anti-inflammatory agents, such as acetaminophen, aspirin, aminopyrine, phenylbutazone, mefanic acid, ibuprofen, diclofenac sodium, indomethacin, colchicine and probenecid; enzymatic anti-inflammatory agents, such as chymotrypsin and bromelain seratiopeptidase; anti-histaminic agents, such as dephenhydramine hydrochloride, chloropheniramine maleate and clemastine; anti-tussive-expectorants, such as codeine phosphate and isoproterenol hydrochloride; analgesics, such as opioids (like diamorphine, morphine and its polar metabolites, such as morphine-6-glucuronides and morphine-3-sulphate); anti-emetics, such as metoclopramide, ondansetron, chlorpromazine; drugs for treatment of epilepsy, such as clonazepam; drugs for treatment of sleeping disorders, such as melatonin; drugs for treatment of asthma, such as salbutamol.

In some embodiments, the therapeutic agent comprises an antisense oligonucleotide, e.g. a nucleic acid molecule that comprises or consists of an siRNA or an shRNA. In some embodiments, the therapeutic agent is a polar compound. Polar compounds are those with a partition coefficient (octanol/water system) of less than 50.

The diagnostic agents in the methods and compositions of the invention are not limited to any particular diagnostic agent. In some embodiments, the diagnostic agents comprise agents that may be visualized or detected once they are administered to a mammal. Diagnostic agents may be useful for labeling particular tissues in the mammal, including healthy and diseased tissues. In some embodiments, the diagnostic agent is released from the delivery vehicles or the nano/microparticles, while in other embodiments they are retained therein to allow visualization of the delivery vehicles or of the nano/microparticles themselves. In some embodiments, the diagnostic agents comprise radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), or magnetic agents, such as gadolinium chelates.

In some embodiments, the diagnostic agent used in the invention is a paramagnetic, radioactive or fluorogenic ion that is detectable upon imaging. In some embodiments, the diagnostic agent is a paramagnetic ion is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). In other embodiments, the diagnostic agent is a radioactive ion such as iodine123, technetium99, indium111, rhenium188, rhenium186, copper67, iodine131, yttrium90, iodine125, astatine-211, and gallium67. In other embodiments, the diagnostic agent is an X-ray imaging agent such as lanthanum (III), gold (III) lead (II) and bismuth (III).

### VII. Therapeutic/Diagnostic Methods

Another aspect of the invention provides methods for the improved transport of therapeutic agents across mucosal surfaces (which includes the presentation of vaccines to mucosal surfaces) in mammals, and a method of treating a human or other mammal comprising administering a composition, comprising at least one of the delivery vehicles described herein, preferably to a mucosal surface of that human or other mammal. Mucosal membranes include, but are not limited to, mucosal membranes in the vagina, buccal cavity, rectum, lungs, eye, colon, small intestine, stomach or nasal cavity.

Some aspects of the invention provide methods and reagents for treating/preventing/diagnosing or aiding in the treatment/prevention/diagnosis of disorders in an individual in need thereof. The invention also relates, in part, to methods and reagents for prognosing or aiding in the prognosing of an individual diagnosed with a disorder.

Some aspects of the invention provide methods of increasing the serum concentration of a therapeutic agent in an individual in need thereof. Preferred methods include administering a composition comprising a plurality of delivery vehicles, the delivery vehicles comprising a therapeutic/diagnostic agent, to a mucosal membrane of the individual, wherein the delivery vehicles cross the mucosal membrane and enter the bloodstream where the therapeutic agent is released. In preferred embodiments, the individual is afflicted with a disorder that is treatable with, or at least one of the symptoms of the disorder is alleviated with, the therapeutic agent, or a disorder that may be diagnosed with the diagnostic agent. These methods are not limited to any particular disorder. In another preferred embodiment, the agent whose concentration in the serum is increased is a polypeptide or peptide.

The therapeutic compositions according to the invention may be administered to the individual, for example, orally, nasally, vaginally, buccally, rectally, via the eye, or via the pulmonary route, in a variety of pharmaceutically acceptable dosing forms, which will be familiar to those skilled in the art.

For example, the delivery vehicles of the invention may be administered via the nasal route using a nasal insufflator device. Example of these are already employed for commercial powder systems intended for nasal application (e.g. Fisons Lomudal System). Details of other devices can be found in the pharmaceutical literature (see for example Bell, A. Intranasal Delivery devices, in Drug Delivery Devices Fundamentals and Applications, Tyle P. (ed), Dekker, New York, 1988).

The delivery vehicles can be administered to the vagina in a freeze dried powder formulation. Microspheres for example may be administered in a vaginal applicator and once in the vagina, the microspheres are released by pressing a syringe-type piston or similar release mechanism on the applicator. Alternatively, the delivery vehicles may be formulated as a powder using a powder device, formulated into a vagina suppository or pessary or vaginal tablet or vaginal gel.

The delivery vehicles can also be administered to the eye in a gel formulation. For example, before administration, nano/microspheres may conveniently be contained in a two compartment unit dose container, one compartment containing the freeze-dried microsphere preparation and the other compartment containing normal saline. Prior to application, the two compartments are mixed and a gel is formed, which is then administered to the eye.

Other delivery routes include via the pulmonary route using a powder inhaler or metered dose inhaler, via the buccal route formulated into a tablet or a buccal patch, via the rectal route formulated into suppositories; via the eye in the form of a powder or a dry ointment, and via the oral route in the form of a tablet, a capsule or a pellet (which compositions may administer agent via the stomach, the small intestine or the colon), all of which may be formulated in accordance with techniques which are well known to those skilled in the art.

In preferred embodiments, the therapeutic/diagnostic agent is a polypeptide. In certain embodiments, the therapeutic methods comprise the step of coadministering to the individual an agent which increases the general permeability of the mucosal surface. In certain other preferred embodiments, the therapeutic methods do not comprise the step of coadministering to the individual an agent which increases the general permeability of the mucosal surface. Such agents generally act by either permeabilizing the mucus layer by degrading/modifying its components, or by permeabilizing the biological membranes of the epithelium under the mucus layer.

Compounds which increase the permeability of the mucus layer include mucolytic compounds such as detergents, sulflydryl compounds and mucolytic enzymes. Exemplary mucolytic detergents include Tween (polyoxyethylenesorbitan) Triton-X100 (t-octylphenoxypolyethoexyethanol) and ambroxol (2-amino-3,5-dibromo-N-[trans-4-hydroxycyclohexyl]benzylamine). Exemplary mucolytic sulfhydryl compounds include N-acetyl cysteine and dithiothreitol (DTT). Exemplary mucolytic enzymes include pronase, papain, bromelain and trypsin.

Compounds which increase the permeability of mucosal membranes include compounds that interact with lipids and/or proteins on the cell membrane of the epithelial cells. Such compounds include but are not limited to, micelles, middle chain fatty acids, salicylic acids and acyl carnitine, EDTA and other calcium/magnesium chelators, sodium caprate, bile salts, the zot toxin from Vibrio cholerae, cholate, reduced glutathione (GSH), polyoxyethylene-24-cholesterol ether, sodium taurodihydrofusidate, phosphato-dihydrofusidate, sodium glycodeoxycholate, sodium salicylate, n-Lauryl-β-D-maltopyranoside and phospholipids.

In certain embodiments, the therapeutic methods comprise the step of coadministering to the individual a protease inhibitor. In other embodiments, the therapeutic methods do not comprise the step of coadministering to the individual a protease inhibitor.

In preferred embodiments, the plurality of delivery vehicles that are administered to the individual comprise both microspheres and nanospheres. In some embodiments, the ratio of microspheres to nanospheres, by weight, is from about 1:5 to about 5:1. In certain embodiments, the weight/weight ratio of nanospheres to microspheres is at least about 0.001, 0.005, 0.01, 0.05, 0.1, 0.2, 0.3, 0.4, 0.5, 0.6, 0.7, 0.8, 0.9, 1, 1.5, 2, 5, 10, 20, 50, 100, 200, 500 or 1000. In one embodiment wherein the compositions comprise both nanospheres of microspheres, the microspheres are greater than 1 μm in diameter and the nanospheres are less than 1 μm. In another embodiment, the composition comprises microspheres and nanospheres, wherein the difference between the average size of the microspheres and the average size of the nanospheres differs by at least 0.1, 0.2, 0.3, 0.5, 1, 1.5, 2, 3, 4, 5, 6, 7, 8, 9 10, 15 or 20 μm. In another embodiment, the composition comprises microspheres and nanospheres, wherein the composite distribution of their size in the composition is a mixture-Gaussian distribution comprising two Gaussian distributions each having its own mean, wherein the two means differ from each other by least 0.1, 0.2, 0.3, 0.5, 1, 1.5, 2, 3, 4, 5, 6, 7, 8, 9 10, 15 or 20 μm.

The nanospheres, microspheres, neither, or both, that are administered to the subject may be have a double coat of a hydrophobic agent and a hydrophilic agent, preferably with the hydrophobic coat(s) applied prior to the hydrophilic coat(s), and preferably both polymers, surrounding a polymeric micro/particle as described in the instant invention. In preferred embodiments of therapeutic methods in which nanospheres are used singly or in combination with microspheres, the nanospheres contain the hydrophobic/hydrophilic double coat.

Section VI of the instant specification lists therapeutic agents each of which, without limitation, may be administered alone or in combination to an individual according to the therapeutic methods provided by the invention. Some classes of preferred therapeutic agents include antisense oligonucleotides (e.g. siRNAs or shRNAs), antibodies, photosensitizers, androgen, estrogen, nonsteroidal antiinflammatory agents, antihypertensive agents, analgesic agents, antidepressants, antibiotics, anticancer agents, anesthetics, antiemetics, antiinfectants, contraceptives, antidiabetic agents, steroids, anti-allergy agents, chemotherapeutic agents, anti-migraine agents, agents for smoking cessation, anti-viral agents, immunosuppresants, thrombolytic agent, cholesterol-lowering agents and anti-obesity agents.

In some embodiments of the methods described herein, the therapeutic compositions are administered to an individual in combination with other treatments. The individual may receive these additional treatments prior to, concurrently with, or subsequently to the administration of the delivery vehicles, multiple unit carrier systems or nano/microparticles. For example, an individual afflicted with breast cancer may be treated with a cocktail of microspheres and nanospheres containing the anti-her2 antibody herceptin while concurrently undergoing surgical procedures, chemotherapy, radiation therapy or hormonal therapy.

Toxicity and therapeutic efficacy of the agents and compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from the cell culture assays and animal studies can be used in formulating a range of dosage for use in humans. Example 19 illustrate the administration of an insulin containing cocktail of nanospheres and microspheres that may be used to test dosage regimens in a mammal. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to determine more accurately useful doses in mammal including humans. Levels of the desired therapeutic or diagnostic agent delivered to the plasma may be measured, for example, by high performance liquid chromatography.

In one embodiment of the methods described herein, the effective amount of the agent is between about 1 mg and about 50 mg per kg body weight of the individual. In one embodiment, the effective amount of the agent is between about 2 mg and about 40 mg per kg body weight of the individual. In one embodiment, the effective amount of the agent is between about 3 mg and about 30 mg per kg body weight of the individual. In one embodiment, the effective amount of the agent is between about 4 mg and about 20 mg per kg body weight of the individual. In another embodiment, the effective amount of the agent is between about 5 mg and about 10 mg per kg body weight of the individual. Examples 20 and 21 exemplify dosage regimens of insulin and recombinant human growth factors for humans.

In one embodiment of the methods described herein, the agent is administered at least once per day. In another embodiment, the agent is administered daily. In yet another embodiment, the agent is administered every other day, every 6-8 days, or weekly.

As for the amount of the compound and/or agent for administration to the individual, one skilled in the art knows how to determine empirically the appropriate amount. As used herein, a dose or amount would be one in sufficient quantities to either inhibit the disorder, treat the disorder, treat the individual or prevent the individual from becoming afflicted with the disorder or condition to be ameliorated. This amount may be considered an effective amount. One of ordinary skill in the art can perform simple titration experiments to determine what amount is required to treat the individual. The dose of the composition of the invention will vary depending on the individual and upon the particular route of administration used. In one embodiment, the dosage may range from about 0.1 to about 100,000 ug/kg body weight of the individual. Based upon the composition, the dose can be delivered continuously, such as by continuous pump, or at periodic intervals. For example, on one or more separate occasions. Desired time intervals of multiple doses of a particular composition can be determined by one skilled in the art.

The effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The effective amount will be known to one of skill in the art; it will also be dependent upon the form of the compound, the size of the compound and the bioactivity of the compound. One of skill in the art may routinely perform empirical activity tests for a compound to determine the bioactivity in bioassays and thus determine the effective amount. In one embodiment of the above methods, the effective amount of the compound comprises from about 1.0 ng/kg to about 100 mg/kg body weight of the individual. In another embodiment of the above methods, the effective amount of the compound comprises from about 100 ng/kg to about 50 mg/kg body weight of the individual. In another embodiment of the above methods, the effective amount of the compound comprises from about 1 μg/kg to about 10 mg/kg body weight of the individual. In another embodiment of the above methods, the effective amount of the compound comprises from about 100 μg/kg to about 1 mg/kg body weight of the individual.

As for when the compound, compositions and/or agent is to be administered, one skilled in the art can determine when to administer such compound and/or agent. The administration may be constant for a certain period of time or periodic and at specific intervals. The compound may be delivered hourly, daily, weekly, monthly, yearly (e.g. in a time release form) or as a one time delivery. The delivery may be continuous delivery for a period of time, e.g. intravenous delivery.

### EXEMPLIFICATION

One skilled in the art would readily appreciate that the present disclosure is well adapted to carry out the objectives and obtain the ends and advantages mentioned, as well as those inferred herein. The chitosan polymer modified (SparkCORAL™) microspheres along with the method, procedures, treatments, molecules and specific compounds described herein are exemplary and are not intended as limitations on the scope of the disclosure. Changes therein and other uses will occur to those skilled in the art which are encompassed within the spirit of the disclosure are envisioned to fall within the scope of the claims.

It will be readily apparent to one skilled in the art that varying substitutes and modifications may be made to the disclosed inventions therein without departing from the scope and spirit of the disclosure.

The contents of any patents, patent applications, patent publications, or scientific articles referenced anywhere in this application are herein incorporated by reference in their entirety.

In order that this invention may be better understood, the following examples are set forth. These examples are for purposes of illustration only and are not to be construed as limiting the scope of the invention in any manner. For example, although some of the examples below describe the coating or loading of therapeutics into microspheres, similar protocols may be suitably applied to nanospheres, and vice versa.

### Example 1

### Synthesis of Chitosan Microspheres

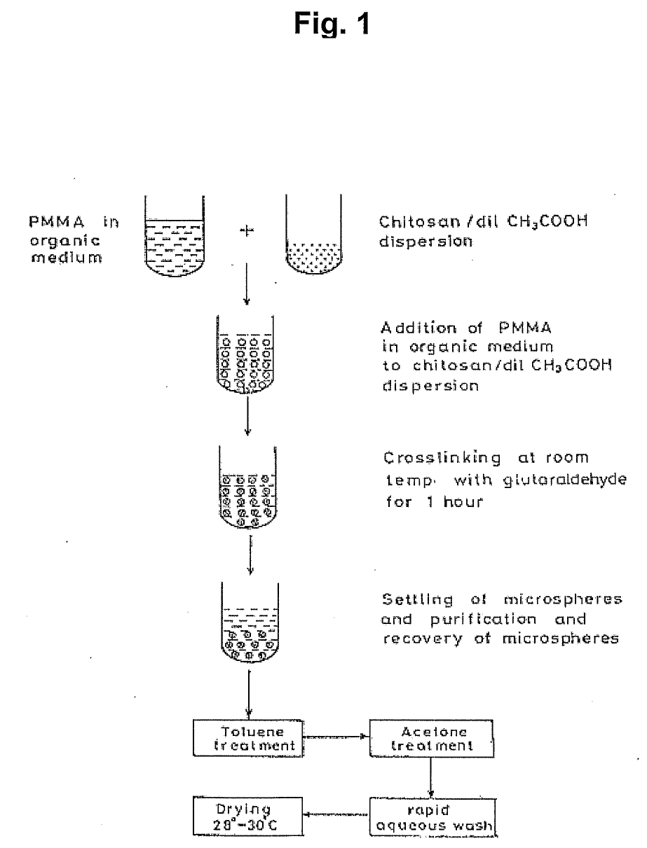

Bovine Serum Albumin (BSA) was chosen as a model protein because it is well characterized, readily soluble in water, and large in size (molecular weight=68,000 Dalton). BSA (Sigma, USA), polycaprolactone (Aldrich, USA), methyl methacrylate (MMA)(SRL, India) were used in the polymerization studies. Chitosan (Sigma, USA), Glutaraldehyde 25% (Flukon, AG) were used in the preparation of microspheres. An exemplary general method of preparing chitosan microspheres is diagrammed in FIG. 1. A 5 ml solution of 0.05 molar acetic acid containing 2% chitosan solution was dispersed in 20 ml of 10% poly methyl methacrylate (PMMA) solution (toluene:chloroform, 1:1, v/v) by stirring at 8000 rpm, using a standard stirrer.

After 5 minutes of dispersion, 7.5 ml of glutaraldehyde-saturated toluene was added to the chitosan solution to induce cross-linking while stirring at 8000 rpm. The stirring was continued for 3 minutes after the addition of glutaraldehyde. The crosslinking of chitosan was allowed to proceed for 1 hour to overnight depending on the degree of crosslinking desired.

The cross-linked microspheres were retrieved after sedimentation by decanting and were then washed with toluene (5 times) followed by an acetone wash (5 times) using 20 ml solvent each time to remove PMMA. The microspheres were given a final wash with distilled water, dried at room temperature and stored as a powder at 4° C.

### Example 2

### Synthesis of Chitosan Nanospheres

Chitosan nanospheres were prepared by using similar polymer dispersion techniques as described above for the preparation of chitosan microspheres, except for a few changes in experimental conditions. These changes include use of 0.5% chitosan in 0.05 M acetic acid. Other concentration ranges of chitosan such as, for example, 0.2-2.0% can also be used. The poly methyl methacrylate (PMMA) concentration used was 20%. The concentration of PMMA can vary depending upon the particle size. For example, 25% PMMA can be used to obtain smaller nanospheres. The stirring speed was 20,000 rpm. The stirring speed can vary between 5000-25000 rpm depending upon the desired particle size, with greater speeds yielding smaller particles. The glutaraldehyde-saturated toluene used for cross-linking was 7.5 ml. The chitosan nanospheres were centrifuged at 10,000 rpm after acetone washes and dried under vacuum at room temperature. The dried nanosphere powder was stored at 4° C. until further use.

### Example 3

### Loading of Chitosan Microspheres with Therapeutic Agents

Proteins were incorporated into chitosan microspheres by two methods, namely by swelling (indirect) or during preparation (in situ or direct). In the exemplary method below, BSA was used as a model protein.

(a) Direct Method: A 2% solution of chitosan in aqueous acetic acid (0.05M) containing various quantities (5 mg, 10 mg, 25 mg, 50 mg) of BSA was dispersed in a 10% PMMA solution by stirring as described herein under preparation of chitosan microspheres. Various quantities of BSA were used to monitor the in vitro release pattern of BSA from the microspheres under simulated conditions (e.g., at pH 7.4 to simulate intestinal pH). The microspheres were cross-linked with glutaraldehyde-saturated toluene as described herein. The BSA containing chitosan microspheres were retrieved after sedimentation by washing with toluene as described above in the microsphere preparation description. The microspheres were finally washed with a phosphate buffer (pH 7.4) to remove any surface adsorbed BSA.

(b) Indirect Method: The incorporation of protein (BSA) was done into the preformed microspheres by incubating the microspheres in an aqueous BSA solution. 100 mg of microspheres were suspended in an aqueous solution containing 25 mg BSA (approximately 10 ml) in phosphate buffer (pH 7.4) and left overnight. The microspheres containing BSA were recovered by centrifuging and washing quickly (to remove the surface adsorbed BSA) with phosphate buffer three times. Microspheres were dried and stored at 4° C. for further use.

Similar protein loading methods were used to prepare chitosan nanospheres containing BSA. The concentrations of chitosan, PMMA, and the stirring speed were adjusted as described in the chitosan nanosphere preparation method of Example 2.

Entrapment efficiency of drugs in chitosan microparticles was determined using the following formula: Entrapment Efficiency (%)=(Total Drug−Free Drug)/Total Drug; wherein entrapment efficiency=how much of the drug is entrapped as a %; total drug is the drug amount used during entrapment; and free drug is the drug amount remaining in the solution after entrapment. Loading efficiency of drugs in chitosan microparticles, or how much Drug is in the microspheres as a %, was determined using the following formula: Loading Efficiency (%)=(Total Drug−Free Drug)/Microspheres weight.

A similar direct or indirect method is used to load nanospheres with BSA, insulin, or other therapeutic/diagnostic agents.

### Example 4

### Coating of Chitosan Microspheres with Hydrophobic Polymers

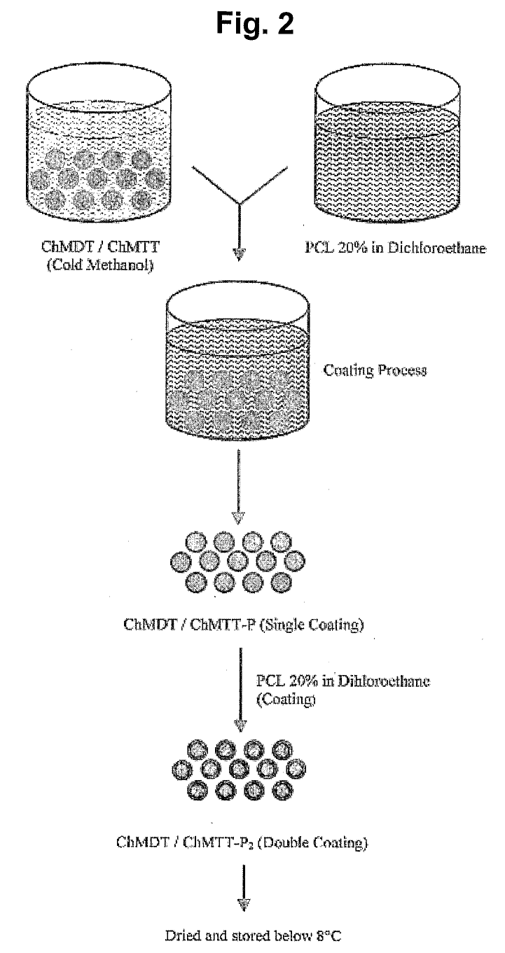

Chitosan Microspheres were coated with polycaprolactone to generate SparkCORAL™ microspheres. Briefly, chitosan microspheres loaded with model protein BSA were coated with PCL to impart hydrophobicity and also to extend the release of the entrapped drug. A known quantity of microspheres (e.g, 100 mg) was allowed to swell in a sufficient quantity of methanol (e.g., 10 ml) at 4° C. for 30 minutes and then coating was achieved by decanting the methanol and immediately adding a solution of cold PCL using 5%, 10% or 20% w/v PCL in acetone or dichloroethane (FIG. 2). Microspheres with additional coats of PCL were prepared in a similar manner. For this two sets of chitosan microspheres were coated with 5% and 10% w/v PCL initially and then were coated with a subsequent coat of PCL having 5% and 10% w/v concentrations respectively. The resulting PCL coated SparkCORAL™ microspheres were strained, air-dried and stored at 4° C.

### Example 5

### Morphological Examination of PCL-Coated and Non-Coated Chitosan Microspheres by Electron Microscope

Scanning electron microscope (SEM) indicates that the placebo chitosan microspheres appeared spherical, uniform with a smooth surface. PCL coating does not alter the morphology except to show the surface coating of polymer. Dramatic changes of chitosan microsphere surface characteristics occurred with the release of drug with time. The surface of the microspheres changed from smooth and spherical to rough, non-spherical and vacuolated. The initial intact surface of the polymer coating was severed by the release of the entrapped protein, which diffused out through the polymer layer in the course of time (FIG. 3). PCL coating provided a barrier by blocking the pores of the swollen microspheres preventing easy release of the entrapped protein.

### Example 6

### Examination of PCL-Coated and Non-coated Chitosan Microspheres by Infrared Spectra

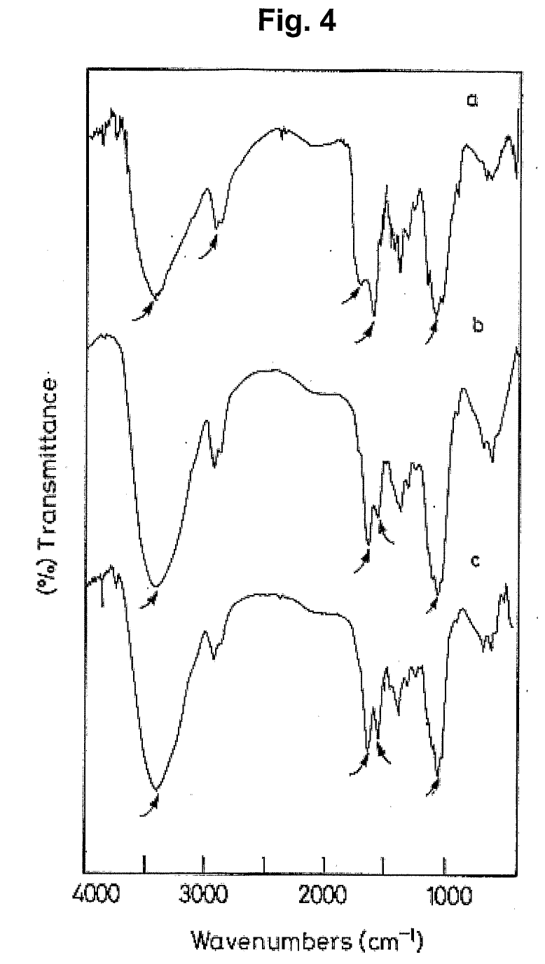

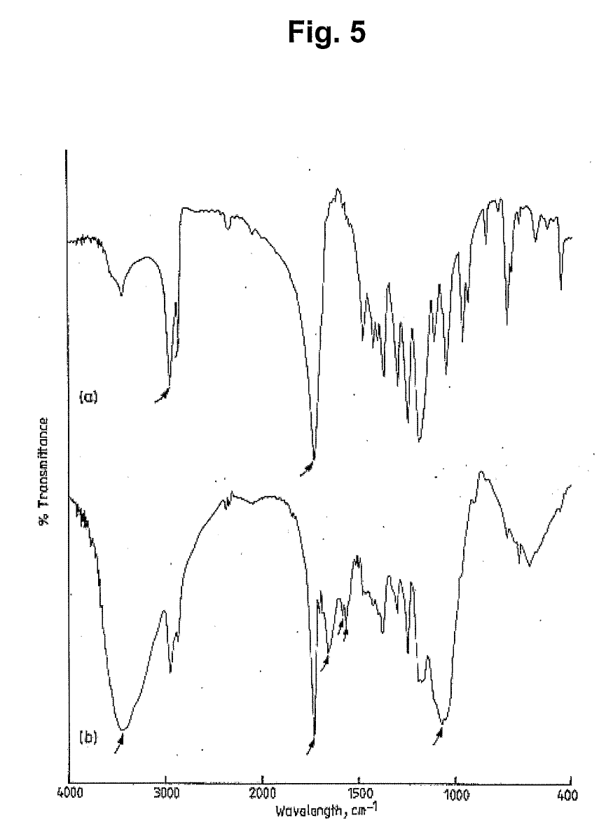

The infrared spectra of microspheres indicated the structural characteristics of chitosan, chitosan microspheres and PCL coated chitosan microspheres. Infrared spectrum of chitosan shows an absorption band of 1565 cm⁻¹, which corresponds to the vibration of the NH₂ group. Absorption in the region of 1650 cm⁻¹ corresponds to the carbonyl groups whereas OH stretching frequency observed at 3420 cm⁻¹ (FIGS. 4 and 5). The band at about 2940 cm⁻¹ corresponds to —CH vibration and is assigned to the CH stretching vibration of the pyranose ring. In the case of crosslinked chitosan microspheres, characteristic doublet at 1657 cm⁻¹ and 1580 cm⁻¹ due to the C═N—, a Schiff s base type structure was observed. The BSA loaded chitosan microspheres spectrum showed significant increase in the intensity of the 1565 cm⁻¹ band which was due to the overlapping of NH₂ groups of protein and chitosan. No other characteristic peak was seen, giving the inference of no significant interaction occurring between the functional groups present on the outer surface of crosslinked microspheres and BSA. PCL-coated chitosan microspheres displayed a characteristic absorption band at 1725 cm⁻¹ due to C═O stretching of ester after a lactone ring opening. The infrared spectral absorbance profile of SparkCORAL™ microspheres loaded with BSA displayed bands for chitosan (3420 cm⁻¹), BSA (565 cm⁻¹) and PCL (1725 cm⁻¹) revealed the structural integrity of the microspheres and incorporated contents. The infrared spectral studies demonstrated that BSA was effectively incorporated in the microspheres with a PCL coating (FIGS. 4 and 5).

### Example 7

### Thermal Analysis of PCL-Coated and Non-coated Chitosan Microspheres by Infrared Spectra

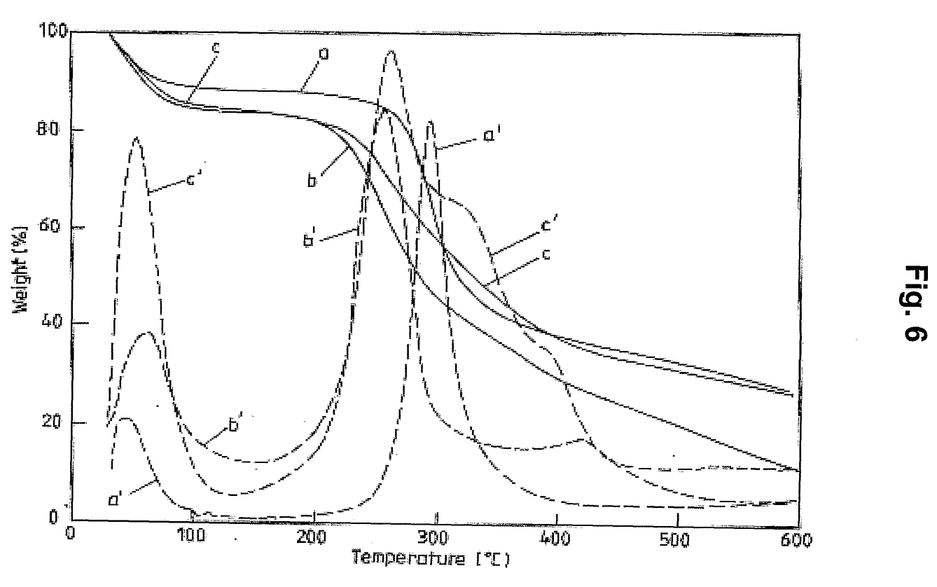

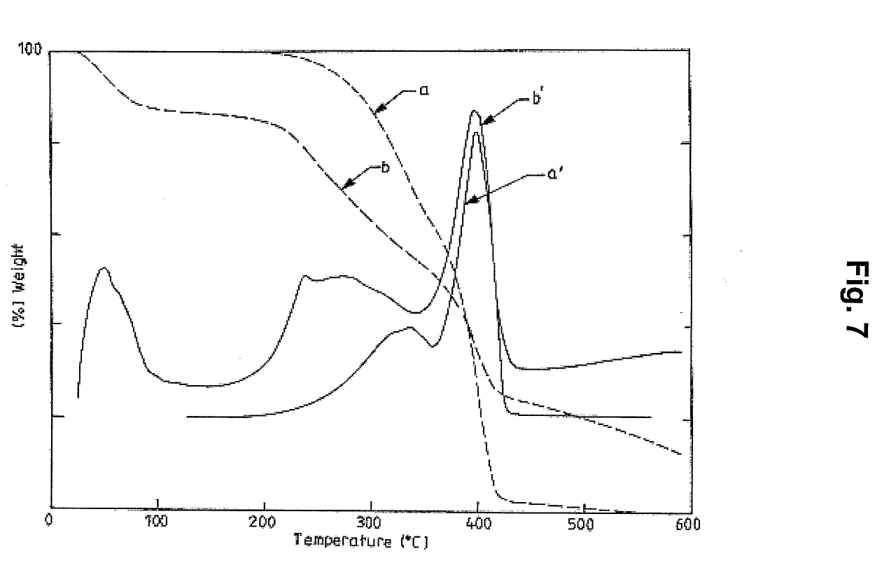

The thermogravimetric analysis of chitosan, chitosan microspheres and chitosan microspheres loaded with BSA showed a first stage 13% weight loss due to the elimination of water molecules (FIG. 6). Maximum decomposition of chitosan occurred at 300° C. However, in the case of chitosan microspheres, maximum decomposition occurred at 265° C. (FIG. 6). The decrease in the thermal stability of chitosan microspheres when it was crosslinked with glutaraldehyde may be due to the replacement of hydrogen atoms from the amino functional groups of chitosan for the formation of Schiff's base type crosslinks. The PCL coated chitosan microspheres showed the maximum decomposition peak at much higher temperature at 410° C., which indicated the increased stability of PCL coated chitosan microspheres (FIG. 7).

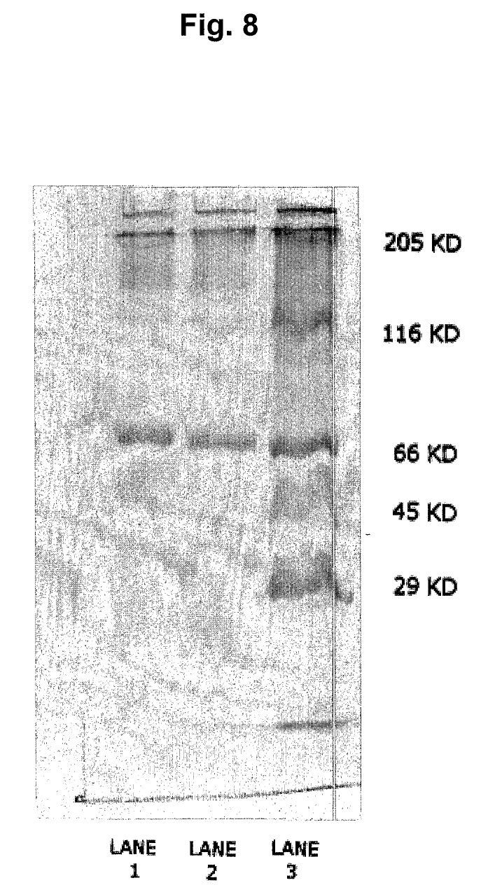

Conformational integrity of entrapped BSA SDS-PAGE analysis was performed to evaluate conformational integrity of BSA during chitosan microsphere formation (FIG. 8). The standard or unencapsulated BSA and the BSA released from the chitosan microspheres were virtually identical. SDS-PAGE of the released protein showed one band that correlated with intact BSA. This clearly indicated that the protein did not react chemically with the matrix material. Moreover, these results also demonstrated that the method adopted for the encapsulation of BSA into the microspheres did not lead to a significant irreversible aggregation or degradation of the carrier macromolecule.

### Example 8

### Quantification and Characterization of Protein Loading

Chitosan microspheres were weighed and digested by 6N HCl hydrolysis for 15 hours at 100° C. The resultant solution was filtered through 0.45 micrometer Millipore filter and the clear filtered solution with an appropriate dilution was read at λmax 640 nanometer after making background correction according to Lowry's protein assay method using a spectrophotometer.

SDS-PAGE analysis: SDS-PAGE analysis was carried out according to the standard method of Laemmli to study the stability of BSA during incorporation and residence within the microspheres.

Morphology and particle size analysis: Particle size distribution of microspheres was analyzed using a particle size analyzer (Malvern Masterizer). The chitosan microspheres were dispersed in HPLC grade water and analyzed for particle size.

The morphological characteristics (shape, size and surface) of the chitosan microspheres were studied using a scanning electron microscope (SEM) (FIG. 3). The microspheres for SEM analysis were prepared by dispersing the dried microspheres into one side of a double adhesive tape which was stuck to an aluminum stub. The stubs were then coated with gold using a Polaron SC500 sputter coater to a thickness of 20-30 nanometers and examined under the scanning electron microscope (Leika Steroscan 5440, U.K) for their structural features.

Infrared spectra and thermal analysis: The infrared spectra of the different stages of the chitosan microsphere formulation was obtained using potassium bromide pellets. The infrared spectra of the samples were assayed using a Fourier transformed infrared spectrometer (Nicolet 20 DXB, Madison, Wis.).

Thermo gravimetric analysis (TGA): Chitosan, chitosan microspheres, BSA loaded chitosan microspheres and PCL-coated BSA loaded chitosan microspheres were analyzed using a Dupont 2000 thermal analyzer with a thermogravimetric module. Dry samples weighing about 3 mg were placed in a pan and a lid was crimped to maximize the contact between the sample and the pan. The samples were heated from room temperature to 600° C. at a constant heating rate of 10° C./min under nitrogen purge.

The amount of BSA entrapped in chitosan microspheres was determined in increasing BSA concentrations by digesting microparticles in 6N HCl. Increasing the amount of BSA concentration from 25 to 50 mg increased the protein loading from 9 to 15%, whereas the percent encapsulation decreased from 68 to 60%.

### Example 9

### In vitro Protein Release Studies from PCL-Coated Chitosan Microspheres In Vitro

Release studies of chitosan microspheres after the incorporation of BSA and polymer coating were carried out in a phosphate buffered saline pH 7.4 at 37° C. Chitosan microspheres (100 mg) were suspended in 100 ml of phosphate buffer. At the regular time intervals, sample aliquots were removed, filtered through a 0.45 Millipore filter and analyzed according to Lowry's protein assay method for BSA content. Equal amounts of the buffer were added every time to mimic the infinite sink conditions of the body. The in-vitro release data indicated that about 71.06% of BSA was released in 19 days in the case of 5% PCL-coated chitosan microspheres. On the other hand, 72.4% of BSA was released in 27 days in the case of 5% PCL double-coated chitosan microspheres. Increasing the coating concentration, with a single coat of 10% PCL resulted in a prolonged release (30 days) of 70.4% of BSA from the microspheres. 5% single PCL-coat chitosan microspheres and 10% single PCL coat chitosan microspheres released approximately the same amount (71.06% and 70.4%) within a period of 19 and 30 days respectively. A double coating of a concentrated PCL solution was more effective in retarding BSA release than a single coating of PCL of lesser concentration. A 20% double coated chitosan microspheres released 70.9% in 66 days compared to 20% single coated chitosan microspheres (71.2% in 48 days).

### Example 10

### Preparation of Chitosan-Insulin (SparkCORAL™) Nano/Microspheres

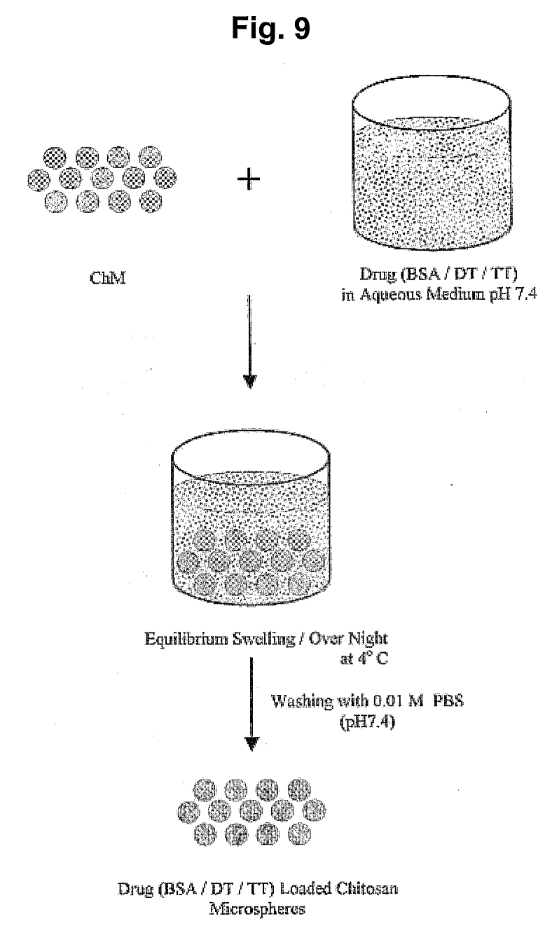

Insulin can be incorporated into the SparkCORAL™ nano/microspheres during the preparation (direct method) or absorbed into the preformed spheres (in-direct method) (FIG. 9).

In the direct method, insulin containing chitosan microspheres (SparkCORAL™) can be prepared in a similar manner as placebo chitosan microspheres except that a solution containing a known quantity of Insulin (80 IU/ml-insulin dissolved in 0.01N HCl at various concentrations) would be added to the chitosan before dispersing using PMMA.

In the indirect method, insulin can be loaded into the pre-prepared SparkCORAL™ microspheres by placing the spheres in a concentrated insulin solution for 6 hours. The insulin diffused into the gel matrix of the spheres and then the pH of the matrix can be raised to neutral pH with 0.01 N NaOH and quickly washed three times with phosphate buffer (pH 7.4). The drug incorporated spheres can be dried under vacuum and stored at 4° C. prior to use.

Enzymes such as glucose oxidase and catalase can also be included in combination with insulin to enhance glucose responsive insulin release. For example, a range of about 100 U/ml to about 1000 U/ml of glucose oxidase and a range of about 100 U/ml to about 1000 U/ml of catalase can be included in the chitosan nano/microspheres either via the direct method or via the indirect method described above for immobilizing insulin.

### Example 11

### Analysis of Insulin Content in SparkCORAL™ Nano/Microspheres

The amount of insulin absorbed on the surface of the microspheres may be determined from the insulin content in the phosphate buffer washings. Aliquots of phosphate washings are filtered through a 0.45 micrometer Millipore filter and the absorption at 272 nanometer can be measured to determine the insulin content. The amount of insulin entrapped in the microspheres can be calculated from the difference between the amount of insulin loaded and the insulin removed from the surface of the spheres. The aliquots from the washings are analyzed spectrophotometrically at 272 nanometers or assayed using reversed phase liquid chromatography (RP-HPLC).

### Example 12

### Preparation of Chitosan-Plasmid DNA Nano/Microspheres

Chitosan nano/microspheres were prepared by the polymer dispersion technique as disclosed herein. The incorporation of plasmid DNA into the preformed spheres by incubating the spheres in aqueous DNA solution. 1 mg of microspheres is suspended in 20 micro liters of plasmid DNA solution in TE buffer (pH 8) and left overnight. The spheres containing DNA may be recovered by washing with 100 micro liters of TE buffer and centrifuged at 2000 rpm for 5 minutes at 4° C. The amount of DNA loading from the retrieved spheres may estimated using ethidium bromide as a flourescent dye and analyzing using a spectroflourimeter. By this method it is expected that the entrapped efficiency may reach as high as 70% or more.

### Example 13

### Effect of Protein Loading on Release Rates of BSA

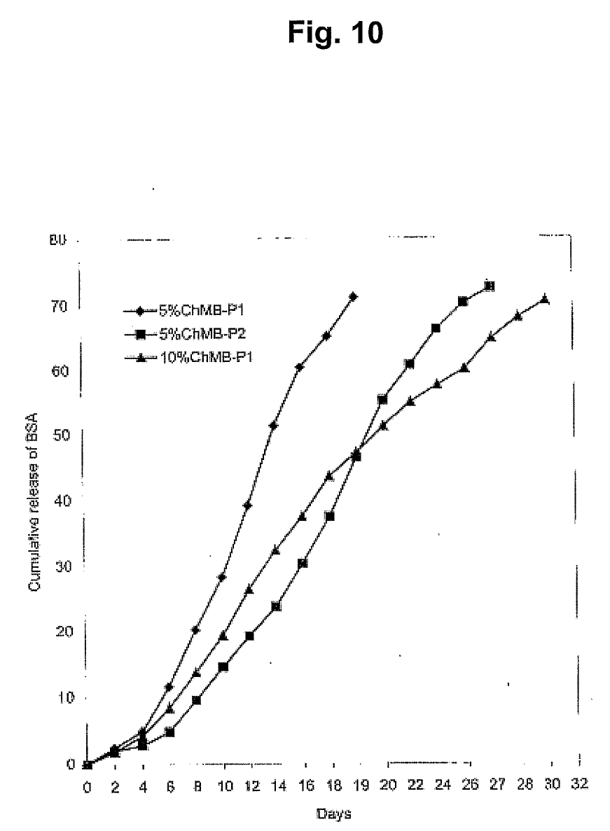

The in-vitro release profiles of chitosan and microspheres loaded with BSA (8.57% and 15.06%) by an indirect method revealed a release rate of BSA (68% in 13 days) which was more efficient in the case of 15.06% protein loaded in chitosan microspheres (FIG. 10). Microspheres loaded with 8.57% released 64.4% in 13 days. These results indicated that the rate of release of BSA increased with protein loading percentage. Higher loading of BSA led to a burst release on day one, in addition to an over-all higher rate of release.

### Example 14

### Measuring Charge per Unit Area/Volume in Aminated Chitosan

The concentration of amino groups in aminated chitosan was measured by using saturation capacity for adsorption of HCl. The chitosan particles were contacted with HCl solution and gently mixed. The amount of HCl adsorbed on the particles was measured after 4 days. The pH of the solution for HCl was analyzed with a pH meter. When the concentration of HCl in the liquid phase was higher than 5 moles/m³, HCl was analyzed with the pH meter. The adsorbed phase concentration of HCl was calculated according the following equation: q=V(C₀−C)/W, where, C₀ and C are the initial concentration and equilibrium concentration of HCl in the liquid phase (kmol/m³), respectively; q denotes the adsorbent phase concentration of HCl (kmol/m³ of wet adsorbent); V and W are the volume of the solution and the wet adsorbent particles (m³), respectively.

In the case of amination using ethylene diamine and glutaraldehyde crosslinked chitosan microspheres, the concentration of amino groups was 8.02 meq/g as assayed using the HCl-based method.

### Example 15

### Loading Chitosan Nanospheres with Therapeutic Agents

Proteins were incorporated into chitosan nanospheres by two methods, namely by swelling (indirect) or during preparation (in situ or direct). In the exemplary method below, insulin was used as the therapeutic agent and incorporation was done by the indirect method. Insulin was incorporated into the preformed nanospheres by incubating the nanospheres in an aqueous insulin solution. Insulin (38 mg) of was dissolved in 1 ml of 0.1N NaOH. 14 ml of cold phosphate buffer (pH 7.2) was added to the solution and shaken well. 1 ml of 0.1 N HCl was added to the solution and shaken well. 150 mg of the nanospheres were added to the solution, shaken and left overnight in refrigerated conditions at 4° C. for insulin incorporation. The nanospheres containing insulin were recovered by centrifuging and washing quickly (to remove the surface adsorbed insulin) with phosphate buffer three times. Nanospheres were dried and stored at 4° C. for further use.

Entrapment efficiency of drugs in chitosan nanoparticles was determined using the following formula: Entrapment Efficiency (%)=(Total Drug−Free Drug)/Total Drug; wherein entrapment efficiency=how much of the drug is entrapped as a %; total drug is the drug amount used during entrapment; and free drug is the drug amount remaining in the solution after entrapment. Loading efficiency of drugs in chitosan nanoparticles, or how much Drug is in the nanospheres as a %, was determined using the following formula: Loading Efficiency (%)=(Total Drug−Free Drug)/nanospheres weight

### Example 16

### Coating of Chitosan Nanospheres with Hydrophobic Polymers

Chitosan nanospheres were coated with polycaprolactone to generate SparkCORAL™ nanospheres. Briefly, chitosan nanospheres loaded with insulin were coated with PCL to impart hydrophobicity and also to extend the release of insulin. A known quantity of nanospheres (e.g, 100 mg) was allowed to swell in a sufficient quantity of ethanol (e.g., 10 ml) at 4° C. for 30 minutes and then coating was achieved by decanting the ethanol and immediately adding a solution of cold PCL using 2.5%, 5% or 10% w/v PCL in benzyl alcohol. Nanospheres with additional coats of PCL were prepared in a similar manner. The resulting PCL coated SparkCORAL™ nanospheres were centrifuged at 4° C. for 10 minutes, air-dried and stored at 4° C.

### Example 17

### Morphological Analysis of Nanospheres

Nanosphere shape and morphology analyzed by Scanning Electron Microscopy (SEM) and Transmission Electron Microscope (TEM). SEM Samples are mounted on aluminum stubs and sputter coated with carbon under reduced pressures. A 30-40 nanometer thick carbon coat was applied using sputter carbon coater. The sample assembly placed in the microscope and vacuum is created. The nanospheres are observed under the scanning electron microscope (Hitachi S4700) at an accelerating voltage of 15 KV. TEM sample preparation is done by suspending the nanospheres in an isopropyl alcohol solution. A syringe is used to place a droplet of the solution on a copper grid. The sample assembly is loaded into the microscope and a vacuum is created. The nanospheres are observed under the transmission electron microscope (Phillips CM30) at a voltage of 100 KV.

### Example 18

### Preparation of Chitosan Nanosphere and Microsphere Cocktails for Insulin Oral Delivery

Chitosan microspheres and nanospheres are prepared according to Examples 1 and 2, respectively. The spheres are optionally aminated and the free and residual or free aldehyde groups are blocked. The spheres are loaded with insulin with or without catalase and glucose oxidase, as described in Example 11. The spheres are coated with PCL, followed by a coating with chitosan. Coating with PCL is described in Example 4. The loading efficiency may be determined using methods such as those described in Example 11. A multiple unit carrier system suitable for oral administration and subsequent transmucosal absorption in the intestine of the microspheres and nanospheres is prepared by loading a cocktail of the microspheres and nanospheres, in a 1:1 w/w ratio, into a gelatin capsule size 9 coated with a pH sensitive polymer. In vivo and in vitro release studies of insulin from the capsule are tested

### Example 19

### In-Vivo Evaluation of Chitosan Nanosphere and Microsphere Cocktails for Insulin Oral Delivery

The following protocol for the studies on animals has been approved by the animal ethical committee of the institution (IAEC) and adheres to the principles of laboratory animal care. Healthy male Wistar rats (200 g-300 g body weight) obtained from Vels Pharmacy College, Chemai, were used. Seven groups of rats, each group containing six rats, were used with six groups as experimental and the seventh group was a non-diabetic control. Plasma glucose levels were measured prior to administration of insulin. Diabetes was induced in rats by intraperitoneal injection of alloxan—100 mg/kg of body weight, dissolved in saline solution. Rats were considered diabetic when fasted glycemia was greater than 250 mgs/dl, 72 hours after alloxan treatment. Oral administration of the insulin was done by gently pushing a dosing syringe through the rats' esophagus. The nature of treatment and dosages are as detailed in the following table:

| Groups | Nature of treatment | Dosage received by each rat |
|---|---|---|
| I | Untreated, diabetic rats | 1 ml saline administered orally |
| II | Placebo microspheres in | Administered orally - One coated capsule |
| | coated capsules | (size 9) containing placebo microspheres |
| | | administered orally by force feeding using |
| | | a dosing syringe, followed by 1 ml of saline |
| | | solution |
| III | Subcutaneous injection of | Subcutaneous injection of human insulin |
| | human insulin | (12.5 IU/kg body weight) dissolved in |
| | | 0.5 ml of saline solution |
| IV | Subcutaneous injection of | Subcutaneous injection of nanospheres and |
| | glucose sensitive | microspheres (1.7 mgs in 0.5 ml saline |
| | nanospheres and | solution) containing human insulin |
| | microspheres containing | (50 IU/kg body weight) |
| | human insulin | |
| V | Oral administration of | One coated capsule (size 9) containing |
| | nanospheres and | nanospheres and microspheres (3-4 mg) |
| | microspheres containing | entrapped with human insulin (100 IU/kg) |
| | human insulin | administered orally by force feeding using |
| | | a dosing syringe, followed by 1 ml of saline |
| | | solution |
| VI | Oral administration of | One coated capsule (size 9) containing |
| | glucose sensitive | nanospheres and microspheres (3-4 mg) |
| | nanospheres and | entrapped with human insulin (100 IU/kg) |
| | microspheres containing | administered orally by force feeding using |
| | human insulin | a dosing syringe, followed by 0.5 ml of |
| | | saline solution. |
| | | Glucose solution (1 g/kg body weight) |
| | | administered orally by force feeding |
| | | immediately after receiving capsules. 2 gms |
| | | of glucose dissolved in 10 ml of water for |
| | | injection (200 mgs/ml). 1 ml of this solution was |
| | | given to each rat. |
| VII | Non-diabetic, normal rats, | — |
| | control | |

Blood samples collected from the retro-orbital plexus of the rats, prior to oral administration to establish baseline glucose levels and at different times—0.5, 2, 4, 6, 8 and 24 hours after dosing. Plasma glucose levels expressed as a percent of the baseline plasma glucose levels established prior to oral administration. Clear supernatant plasma was separated from blood samples by centrifugation (2000 rpm, 5 min) and stored under refrigerated conditions for further analysis. Glycemia was determined in plasma sample by glucose oxidase method using glucose autoanalyzer immediately after blood collection. Plasma insulin levels were measured by employing reverse phase HPLC procedure. Insulin was extracted from plasma into 0.05 NHCl (with an intermediate extraction into organic phase) and injected into a reversed phase (ODS, 5 micron, 25 cm×46 mm id) analytical column with mobile phase 74:26 v/v of 0.2 M sodium sulfate (adjusted to pH 2.3 with phosphoric acid) and acetonitrile. The eluent was monitored at 214 nm at a flow rate of 1.2 ml/min. Plasma insulin concentration was determined from the standard curve of insulin, the peak height versus insulin concentration, prepared over a range of 75-800 micro IU/ml.

### Example 20

### Protocol for Administration of Insulin Formulations to Humans

Gel capsules loaded with a microsphere/nanosphere cocktail are generated according to Example 19 for oral administration to human afflicted with type II diabetes or with sugar intolerance. For an average human with a weight of 70 kg, a normal rate of endogenous insulin secretion in adult human is 0.25 to 1.5 IU/hour. Accordingly, the human would be administered an oral dosage of insulin of about 10-11 mg insulin (300 IU) per day. This may be adminstered as a single gelatin capsule daily containing 200 mg of nanosphere and microsphere cocktail. In combination, subcutaneous injections of insulin may also be administered as needed.

### Example 21

### Protocol for Administration of hGH Formulations to Humans

For a child, 0.35 mg rhGH/kg is needed per week, or about 8.75 mg for a 25 kg child. This corresponds to 8.75 mg for 100 to 200 mg nanosphere and microsphere cocktail needed per day. Accordingly, one gelatin capsule containing 200 mg nanosphere and microsphere cocktail is administered orally per day. For an adult, 0.1 to 0.2 mg rhGH/kg is needed per week, or 14 mg rhGH for a person of 70 kg weight. Thus, 14 mg for 200 to 500 mg nanosphere and microsphere cocktail is administered per day. Accordingly, one gelatin capsule containing 500 mg nanosphere and microsphere cocktail per day is administered to the adult.

1. A method of making delivery vehicles suitable for oral delivery of a therapeutic or diagnostic agent to a mammal, the method comprising
(a) providing polymeric microparticles or nanoparticles comprising a therapeutic or diagnostic agent interspersed therein;
(b) coating the polymeric particles, sequentially, with (i) a hydrophobic agent; and
(ii) a hydrophilic agent.

2. The method of claim 1, wherein the polymeric microparticles or nanoparticles comprise gelatin, alginate, chitosan, modified chitosan, polyamino acids, polypeptides, polycaprolactone/PEG mixture, dextran sulfate, dermatan sulfate, chondroitin sulfate, keratin sulfate, heparin sulfate, collagen, cellulose, elastin, or hyaluronic acid, or mixtures thereof.

3. The method of claim 1, wherein the polymeric microparticles or nanoparticles are chitosan particles, gelatin particles, alginate particles, protenoid particles or polycaprolactone/polyethylene-glycol particles.

4-31. (canceled)

32. A delivery vehicle suitable for oral delivery of a therapeutic or diagnostic agent to a mammal prepared according to the method of claim 1.

33. A composition suitable for oral administration comprising a plurality of delivery vehicles according to claim 32.

34. The composition of claim 33, further comprising a pharmaceutically-acceptable excipient.

35. The composition of claim 33, wherein the composition comprises a plurality of polymeric particles, wherein a plurality of the polymeric particles are microspheres and a plurality of the polymeric particles are nanospheres.

36. A delivery vehicle suitable for oral delivery of a therapeutic or diagnostic agent to a mammal, the delivery vehicle comprising
(a) a polymeric microparticle or nanoparticle comprising the therapeutic or diagnostic agent interspersed therein;
(b) a hydrophobic coating surrounding the polymeric particle; and
(c) a hydrophilic coating surrounding the hydrophobic coating.

37. A multiple unit carrier system suitable for oral delivery of a therapeutic or diagnostic agent to a mammal, the system comprising
(a) a gel capsule; and
(b) a plurality of delivery vehicles, according to claim 36, contained within said gel capsule.

38-41. (canceled)

42. A method of increasing the serum concentration of a therapeutic agent in an individual in need thereof, the method comprising orally administering to the subject a therapeutically-effective amount of a composition comprising the delivery vehicles according to claim 32.

43-46. (canceled)

47. A delivery vehicle suitable for oral delivery of insulin to the bloodstream of a mammal, the delivery vehicle comprising
(a) a chitosan particle comprising insulin interspersed throughout;
(b) a hydrophobic coating surrounding the chitosan sphere; and
(c) a hydrophilic coating surrounding the hydrophobic coating.

48. (canceled)

49. A multiple unit carrier system for oral delivery of insulin to the bloodstream of a mammal, the delivery vehicle comprising
(a) a plurality of chitosan microparticles or nanoparticles comprising insulin, catalase and glucose oxidase interspersed throughout;
(b) a hydrophobic coating surrounding the chitosan spheres;
(c) a hydrophilic coating surrounding the hydrophobic coating; and
(d) a gel capsule containing the plurality of chitosan spheres.

50. The multiple unit carrier system of claim 49, wherein the chitosan particles further comprise catalase and glucose oxidase interspersed throughout.

51. The multiple unit carrier system of claim 50, wherein the gel capsule releases the chitosan spheres in a pH-dependent manner.

52. The multiple unit carrier system of claim 51, wherein the gel capsule is coated with alginate.

53. A method of preparing a plurality of nanospheres or microspheres comprising:
(a) crosslinking chitosan with a dialdehyde; and
(b) blocking residual or free aldehyde groups in the crosslinked chitosan with a blocked amino acid, ethanolamine, an amino PEG or a diamino PEG.

54. A method of preparing a plurality of nanospheres or microspheres comprising:
(a) crosslinking chitosan with a dialdehyde, and
(b) blocking residual or free aldehyde groups in the crosslinked chitosan, wherein the dialdehyde is selected from dialdehyde dextran, dialdehyde starch, alginate dialdehyde, chitosan dialdehyde, glucose dialdehyde, galactose dialdehyde, hyaluronic acid dialdehyde and heparin dialdehyde.

55-58. (canceled)

59. The method of claim 53, comprising the step of (c) further crosslinking the crosslinked chitosan of (b) with an agent which does not react with amino groups.

60-65. (canceled)

66. The method of claim 53, wherein the chitosan is crosslinked with the dialdehyde in the presence of a therapeutic or a diagnostic agent, and wherein the therapeutic or diagnostic agent becomes interspersed throughout the microspheres or the nanospheres.

67-68. (canceled)

69. The method of claim 54, comprising the step of (c) further crosslinking the crosslinked chitosan of (b) with an agent which does not react with amino groups.
